(19) United States
(12) Patent Application Publication
(10) Pub. No.: US 2024/0228456 A1
(43) Pub. Date: Jul. 11, 2024

(54) PROCESS

(71) Applicant: JOHNSON MATTHEY PUBLIC LIMITED COMPANY, London (GB)

(72) Inventors: Johannes SCHRANCK, Paulsboro, NJ (US); Eric SLACK, Paulsboro, NJ (US)

(21) Appl. No.: 18/557,827

(22) PCT Filed: Jun. 13, 2022
(86) PCT No.: PCT/GB2022/051483

§ 371 Date: Oct. 27, 2023

Related U.S. Application Data
(60) Provisional application No. 63/210,735, filed on Jun. 15, 2021.

(51) Int. Cl. C07D 401/06; C07C 1/207; C07C 41/30; C07C 253/30; C07D 213/24; C07D 277/22; C07D 307/36; C07D 333/10; C07D 403/06; C07D 405/06; C07D 409/06; C07D 417/06

(52) U.S. Cl. CPC C07D 401/06; C07C 1/2078; C07C 41/30; C07C 253/30; C07D 213/24; C07D 277/22; C07D 307/36; C07D 333/10; C07D 403/06; C07D 405/06; C07D 409/06; C07D 417/06

(57) ABSTRACT

The invention relates to a process for synthesising organic molecules. The invention provides a process for forming an sp²-sp³ carbon-carbon bond between a first compound comprising a substituted or unsubstituted aromatic group and a second compound comprising a substituted or unsubstituted aromatic group in the presence of a catalyst, water, and a first base. The process may find use in the preparation of active pharmaceutical ingredients.

### FIELD OF THE INVENTION

The present invention relates to a cross-coupling process. More specifically, the present invention relates to a cross-coupling process for forming an sp²-sp³ carbon-carbon bond.

### BACKGROUND OF THE INVENTION

Cross-coupling processes where sp²-sp³ carbon bonds are formed are important in the synthesis of organic compounds, such as natural products and active pharmaceutical ingredients.

Forming sp²-sp³ carbon-carbon bonds is challenging and often requires the use of harsh conditions and may require the use of toxic reagents and/or high catalyst loadings. Active pharmaceutical ingredients must be of the highest purity. Any by-products, unreacted starting materials, or catalyst (such as heavy metal catalysts) must be removed. Consequently, reactions which employ harsh conditions may not be suitable for use in the formation of active pharmaceutical ingredients, or may require expensive and time consuming purification steps.

7-azaindoles, and derivatives thereof, are important organic subunits which are found as pharmacophores in a number of active pharmaceutical ingredients. Reactions to functionalise 7-azaindoles are therefore of great interest in the pharmaceutical field.

Eur. J. Org. Chem. 2019, 8, 1801-1807, describes the formation of an sp²-sp³ carbon-carbon bond by reacting a (hetero)benzylic alcohol with a (hetero)arylboronic acid. The reaction is carried out in the presence of SO₂F₂, triethylamine and high loadings of a palladium catalyst of up to 5 mol %.

ACS Catal. 2017, 7, 2, 1108-1112 describes a Suzuki-Miyaura cross-coupling reaction using fluorinated sulfone derivatives.

J. Org. Chem. 2014, 79, 12, 5921-5928 describes using a specific palladium-ligand catalyst to form an sp²-sp³ bond in a coupling reaction between a chloromethyl(hetero)arene and a (hetero)arylboron compound.

There remains a need for safer, more environmentally friendly processes of producing sp²-sp³ bonds, in particular for use in the preparation of active pharmaceutical ingredients.

### SUMMARY OF THE INVENTION

In a first aspect, the present invention provides a process for forming an sp²-sp³ carbon-carbon bond, the process comprising:
providing a compound of Formula I

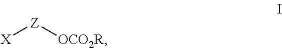

wherein X is a substituted or unsubstituted aromatic group;
Z is an organic group comprising an sp³-hybridised carbon atom C* and having formula —C*(H)(R³)— where R³ is H, OH, a substituted or unsubstituted alkyl group, a substituted or unsubstituted alkenyl group, or a substituted or unsubstituted aromatic group; and
R is a substituted or unsubstituted C₁₋₂₀ straight-chain or C₃₋₂₀ branched-chain alkyl group; and
reacting the compound of Formula I with a compound of Formula II

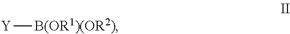

wherein Y is a substituted or unsubstituted aromatic group, and
R¹ and R² are, independently, H or a substituted or unsubstituted organic group comprising 1-20 carbon atoms; or, together with the atoms to which they are attached, R¹ and R² form a ring;
in the presence of a catalyst, water, and a first base, and optionally a Lewis acid, to give a compound of Formula III:

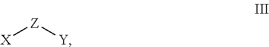

wherein X, Z and Y are as hereinbefore defined; and
wherein:
in the compound of Formula II, the boron atom is bonded to Y at an sp²-hybridised carbon atom in Y; and
in the compound of Formula III, the sp³-hybridised carbon atom C* of Z, is bonded to Y at said sp²-hybridised carbon atom in Y.

It has surprisingly been found that the process of the present invention allows the efficient cross-coupling of an aromatic group with another aromatic group via an sp³-hybridised carbon bridge. The process proceeds in good yields, under mild conditions, and without the need to use aggressive chemical reagents. Such couplings are known to be difficult to achieve, in particular at catalyst loadings acceptable in the pharmaceutical industry.

In a second aspect of the invention there is provided a process for providing the compound of Formula I by reacting a compound of Formula IV,

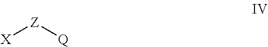

wherein X and Z are as hereinbefore defined; and
Q is hydroxy, or —OM where M is a metal;
with a dialkylcarbonate of formula RO(CO)OR, wherein each R is a substituted or
unsubstituted C₁₋₂₀ straight-chain or C₃₋₂₀ branched-chain alkyl group, in the presence of a second base.

In general, using a compound of Formula IV to prepare the compound of Formula I has the advantage of using a non-toxic, environmentally friendly reagent (i.e. a dialkylcarbonate) which reacts with a compound of Formula IV to give a compound of Formula I. Furthermore, a compound of Formula IV may be more easily sourced than a compound of Formula I, for instance a compound of Formula IV may be more easily sourced from a commercial supplier.

In a third aspect of the invention there is provided an in-situ process of the invention. In the in-situ process of the third aspect of the invention the compound of Formula I is provided in-situ in the presence of the compound of Formula II, the catalyst, the water, and the first base by reacting a compound of Formula IV

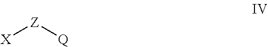

wherein X and Z are as hereinbefore defined; and
Q is hydroxy, or —OM where M is a metal;
with a dialkylcarbonate of formula RO(CO)OR, wherein each R is a substituted or unsubstituted C₁₋₂₀ straight-chain or C₃₋₂₀ branched-chain alkyl group.

Advantageously, the in-situ process of the third aspect of the invention means a compound of Formula I does not have to be supplied, as such, to the reaction of the invention. Instead, the compound of Formula I is formed in-situ from a compound of Formula IV. A compound of Formula IV may be more easily sourced than a compound of Formula I, for instance a compound of Formula IV may be more easily sourced from a commercial supplier. In general, using a compound of Formula IV to prepare the compound of Formula I has the advantage of using a non-toxic, environmentally friendly reagent, a dialkylcarbonate, which reacts with a compound of Formula IV to give a compound of Formula I. It has been surprisingly found that when a compound of Formula I is provided in-situ from a compound of Formula IV that no, or minimal, by-products are formed.

### Definitions

The point of attachment of a moiety or substituent is represented by “—”. For example, —OH is attached through the oxygen atom.

“Alkenyl” refers to a straight-chain or branched unsaturated hydrocarbon group comprising at least one carbon-carbon double bond. An alkenyl group may be substituted by other organic groups, such as an alkyl group, as defined hereinbelow. Typical examples of compounds comprising an alkenyl group include but are not limited to propenyl, but-1-enyl, but-2-enyl, pent-1-enyl, pent-2-enyl, pent-3-enyl, 3,3-dimethylbut-1-enyl, and the like.

“Alkyl” refers to a straight-chain or branched saturated hydrocarbon group. In certain embodiments, the alkyl group may have from 1-20 carbon atoms, in certain embodiments from 1-15 carbon atoms, in certain embodiments, 1-8 carbon atoms. The alkyl group may be unsubstituted. Alternatively, the alkyl group may be substituted. Unless otherwise specified, the alkyl group may be attached at any suitable carbon atom and, if substituted, may be substituted at any suitable atom. Typical alkyl groups include but are not limited to methyl, ethyl, n-propyl, iso-propyl, n-butyl, iso-butyl, sec-butyl, tert-butyl, n-pentyl, n-hexyl and the like.

“Aryl” refers to an aromatic carbocyclic group. The aryl group may have a single ring or multiple condensed rings. In certain embodiments, the aryl group can have from 6-20 carbon atoms, in certain embodiments from 6-15 carbon atoms, in certain embodiments, 6-12 carbon atoms. The aryl group may be unsubstituted. Alternatively, the aryl group may be substituted. Unless otherwise specified, the aryl group may be attached at any suitable carbon atom and, if substituted, may be substituted at any suitable atom. Examples of aryl groups include, but are not limited to, phenyl, naphthyl, anthracenyl and the like.

“Coupling” refers to a chemical reaction in which two molecules or parts of a molecule join together (Oxford Dictionary of Chemistry, Sixth Edition, 2008).

“Heteroaryl” refers to an aromatic carbocyclic group wherein one or more carbon atoms are independently replaced with one or more heteroatoms (e.g. nitrogen, oxygen, phosphorus and/or sulfur atoms). The heteroaryl group may be unsubstituted. Alternatively, the heteroaryl group may be substituted. Unless otherwise specified, the heteroaryl group may be attached at any suitable atom and, if substituted, may be substituted at any suitable atom. Examples of heteroaryl groups include but are not limited to thienyl, furanyl, pyrrolyl, imidazolyl, pyrazolyl, thiazolyl, isothiazolyl, oxazolyl, isoxazolyl, triazolyl, thiadiazolyl, thiophenyl, oxadiazolyl, pyridinyl, pyridazinyl, pyrimidinyl, pyrazinyl, benzoxazolyl, benzthiazolyl, benzimidazolyl, indolyl, quinolinyl and the like.

“Substituted” refers to a group in which one or more hydrogen atoms are each independently replaced with substituents (e.g. 1, 2, 3, 4, 5 or more) which may be the same or different. Examples of substituents include but are not limited to -halo, —C(halo)₃, —Rₐ, ═O, ═S, —O—Rₐ, —S—Rₐ, —NRₐR_{b}, —CN, —NO₂, —C(O)—Rₐ, —COORₐ, —C(S)—Rₐ, —C(S)ORₐ, —S(O)₂OH, —S(O)₂—Rₐ, —S(O)₂NRₐR_{b}, —O—S(O)—Rₐ and —CONRₐR_{b}, such as -halo, —C(halo)₃ (e.g. —CF₃), —Rₐ, —O—Rₐ, —NRₐR_{b}, —CN, or —NO₂. Rₐ and R_{b} are independently selected from the groups consisting of H, alkyl, aryl, arylalkyl, heteroalkyl, heteroaryl, or Rₐ and R_{b} together with the atom to which they are attached form a heterocycloalkyl group. Ra and Rb may be unsubstituted or further substituted as defined herein.

“Arylalkyl” refers to an optionally substituted group of the formula aryl-alkyl-, where aryl and alkyl are as defined above.

“Dialkylcarbonate” refers to a compound of general formula RO(CO)OR, wherein each R is a substituted or unsubstituted C₁₋₂₀ straight-chain or C₃₋₂₀ branched-chain alkyl group. Thus, the R groups may be the same or different. The R groups are independently alkyl groups as defined hereinabove. Examples of dialkylcarbonates include dimethylcarbonate, diethylcarbonate, di-iso-propylcarbonate, di-tert-butylcarbonate, methylethylcarbonate, and methyl-iso-propylcarbonate. “Dialkylcarbonate” does not refer to cyclic carbonates such as ethylenecarbonate or propylene carbonate.

“Halogen”, “halo” or “hal” refers to —F, —Cl, —Br and —I.

“Heteroalkyl” refers to a straight-chain or branched saturated hydrocarbon group wherein one or more carbon atoms are independently replaced with one or more heteroatoms (e.g. nitrogen, oxygen, phosphorus and/or sulfur atoms). The heteroalkyl group may be unsubstituted. Alternatively, the heteroalkyl group may be substituted. Unless otherwise specified, the heteroalkyl group may be attached at any suitable atom and, if substituted, may be substituted at any suitable atom. Examples of heteroalkyl groups include but are not limited to ethers, thioethers, primary amines, secondary amines, tertiary amines and the like.

“Heterocycloalkyl” refers to a saturated cyclic hydrocarbon group wherein one or more carbon atoms are independently replaced with one or more heteroatoms (e.g. nitrogen, oxygen, phosphorus and/or sulfur atoms). The heterocycloalkyl group may be unsubstituted. Alternatively, the heterocycloalkyl group may be substituted. Unless otherwise specified, the heterocycloalkyl group may be attached at any suitable atom and, if substituted, may be substituted at any suitable atom. Examples of heterocycloalkyl groups include but are not limited to epoxide, morpholinyl, piperadinyl, piperazinyl, thirranyl, pyrrolidinyl, pyrazolidinyl, imidazolidinyl, thiazolidinyl, thiomorpholinyl and the like.

“dippf” refers to the compound 1,1′-bis(di-iso-propylphosphino)ferrocene.

“RuPhos” refers to the compound 2-dicyclohexylphosphino-2′,6′-diisopropoxybiphenyl.

“B₂pin₂” refers to the compound 4,4,4′,4′,5,5,5′,5′-octamethyl-2,2′-bi-1,3,2-dioxaborolane, also known as bis(pinacolato)diboron.

“Bpin” refers to 4,4,5,5-tetramethyl-1,3,2-dioxaboron, also known as (pinacolato)boron.

“HBpin” refers to pinacolborane.

“Bcat” refers to (catecholato)boron.

“HBcat” refers to catecholborane.

sp², or sp²-hybridized, refers to the mixing of an s atomic orbital and two p atomic orbitals to produce a hybrid orbital having both s and p character. For example, the carbon atoms in a benzene ring are all considered to be sp² hybridised carbon atoms.

sp³, or sp³-hybridized, refers to the mixing of an s atomic organic and three p atomic orbitals to produce a hybrid orbital having both s an p character. For example, the carbon atom in an alkyl group, for example ethane, are considered to be sp³ hybridized carbon atoms.

“Boc” refers to the organic group tert-butyloxycarbonyl, also known as tert-butoxycarbonyl.

### BRIEF DESCRIPTION OF THE DRAWINGS

FIG. 1 shows a general reaction scheme for the process of the invention.

FIG. 2 shows a preferred process according to the present invention showing the reaction to produce the pharmaceutical compound pexidartinib.

### DETAILED DESCRIPTION

Preferred and/or optional features of the invention will now be set out. Any aspect of the invention may be combined with any other aspect of the invention unless the context demands otherwise. Any of the preferred and/or optional features of any aspect may be combined, either singly or in combination, with any aspect of the invention unless the context demands otherwise.

The process of the invention provides a process for forming an sp²-sp³ carbon-carbon bond, the process comprising providing a compound of Formula I

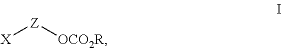

wherein X is a substituted or unsubstituted aromatic group;
Z is an organic group comprising an sp³-hybridised carbon atom C* and having formula —C*(H)(R³)— where R³ is H, OH, a substituted or unsubstituted alkyl group, a substituted or unsubstituted alkenyl group, or a substituted or unsubstituted aromatic group; and
R is a substituted or unsubstituted C₁₋₂₀ straight-chain or C₃₋₂₀ branched-chain alkyl group; and
reacting the compound of Formula I with a compound of Formula II

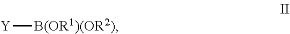

wherein Y is a substituted or unsubstituted aromatic group; and
R¹ and R² are, independently, H or a substituted or unsubstituted organic group comprising 1-20 carbon atoms; or, together with the atoms to which they are attached, R¹ and R² form a ring;
in the presence of a catalyst, water, and a first base, and optionally a Lewis acid, to give a compound of Formula III:

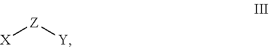

wherein X, Z and Y are as hereinbefore defined; and
wherein:
in the compound of Formula II, the boron atom is bonded to Y at an sp²-hybridised carbon atom in Y; and
in the compound of Formula III, the sp³-hybridised carbon atom C* of Z is bonded to Y at said sp²-hybridised carbon atom in Y.

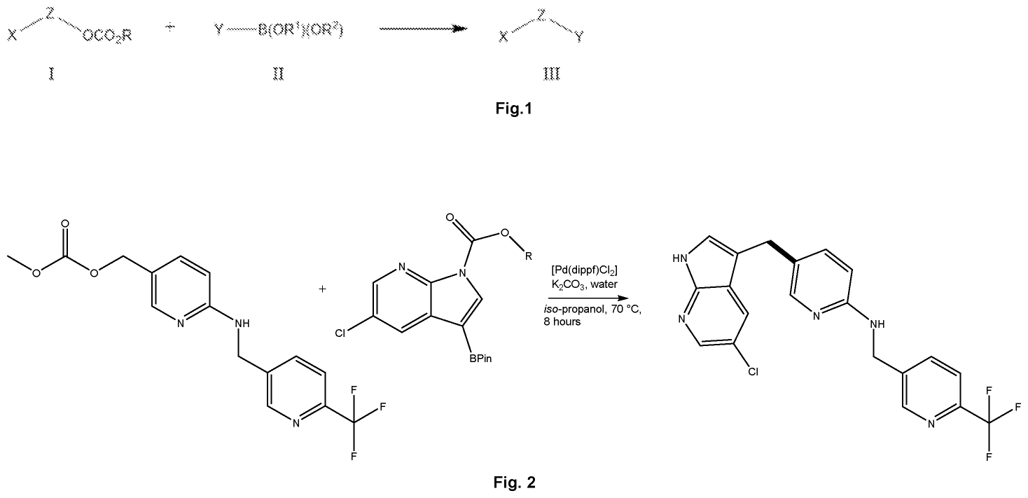

A general reaction scheme for the process of the present invention is depicted in FIG. 1.

In the compound of Formula I, X is a substituted or unsubstituted aromatic group.

In the compound of Formula I, Z is an organic group comprising an sp³-hybridised carbon atom C* and having formula —C*(H)(R³)—. The sp³-hybridised carbon atom C* of Z is bonded directly to the substituted or unsubstituted aromatic group, X. The sp³-hybridised carbon atom C* of Z is also bonded directly to the —OCO₂R moiety (i.e. via the terminal oxygen atom).

In the compound of Formula I, Z is an organic group having formula —C*(H)(R³)— where R³ is H, OH, a substituted or unsubstituted alkyl group, a substituted or unsubstituted alkenyl group, or a substituted or unsubstituted aromatic group. Preferably, R³ is H, OH, or a substituted or unsubstituted aromatic group. More preferably, R³ is H or OH. Most preferably R³ is H.

It may be preferred that R³ is a substituted or unsubstituted aromatic group. For example, it may be preferred that R³ is a substituted or unsubstituted C₆-C₂₀ aryl group, a substituted or unsubstituted C₆-C₁₈ aryl group, or a substituted or unsubstituted C₆-C₁₀ aryl group. Preferred C₆₋₂₀-aryl groups include phenyl, tolyl, xylyl, methoxyphenyl, difluorophenyl, anthracenyl, and naphthalenyl. For example, it may be preferred that R³ is a substituted or unsubstituted C₄-C₂₀ heteroaryl group, a substituted or unsubstituted C₄-C₁₈ heteroaryl group, or a substituted or unsubstituted C₄-C₁₀ heteroaryl group. Preferred heteroaryl groups include pyridinyl, pyrrolyl, imidazolyl, triazolyl, tetrazolyl, indolyl, pyridazinyl, pyrimidinyl, pyrazinyl, quinolinyl, benzimidazolyl, pyrazolyl, azaindolyl, furanyl, benzofuranyl, thiophenyl, benzothiophenyl, thiazolyl, isothiazolyl, thiadiazolyl, 1,2-benzthiazolyl, oxazolyl, isoxazolyl, oxadiazolyl, and benzoxazolyl.

It may be preferred that R³ is a substituted or unsubstituted alkyl group. For example, it may be preferred that R³ is a substituted or unsubstituted C₁ to C₂₀ alkyl group, more preferably a substituted or unsubstituted C₁ to C₁₀ alkyl group (e.g. a C₂ to C₅ alkyl group), most preferably a substituted or unsubstituted C₁ to C₄ alkyl group. R³ may be a straight chain alkyl group or a branched chain alkyl group.

In some processes of the invention R³ and X may be the same as one another (e.g. when R³ is a substituted or unsubstituted aromatic group). In some processes of the invention R³ and X may be different to one another.

In preferred processes of the invention, X in the compound of Formula I is a substituted or unsubstituted aryl group or a substituted or unsubstituted heteroaryl group.

In preferred processes of the invention, X in the compound of Formula I is a substituted or unsubstituted aryl group. Preferably, X in the compound of Formula I is a substituted or unsubstituted C₆-C₂₀ aryl group, more preferably a substituted or unsubstituted C₆-C₁₈ aryl group, even more preferably a substituted or unsubstituted C₆-C₁₀ aryl group. Preferred C₆₋₂₀-aryl groups include phenyl, tolyl, xylyl, methoxyphenyl, difluorophenyl, anthracenyl, and naphthalenyl.

In preferred processes of the invention, X in the compound of Formula I is a substituted or unsubstituted heteroaryl group. Preferably, X in the compound of Formula I is a substituted or unsubstituted C₄-C₂₀ heteroaryl group, more preferably a substituted or unsubstituted C₄-C₁₈ heteroaryl group, even more preferably a substituted or unsubstituted C₄-C₁₀ heteroaryl group. Preferred heteroaryl groups are discussed in more detail below.

In preferred processes of the invention, X in the compound of Formula I is a substituted or unsubstituted heteroaryl group that comprises one, two, three, or four heteroatoms, preferably one or two heteroatoms. The heteroatom may be oxygen, nitrogen, sulfur, or phosphorous, or mixtures thereof. Typically, the heteroatom is nitrogen, sulfur, oxygen, or a mixture thereof. When X in the compound of Formula I comprises two or more heteroatoms, the heteroatoms may be the same or different.

In preferred processes of the invention, X in the compound of Formula I is a substituted or unsubstituted heteroaryl group that comprises one or more nitrogen atoms. For example, X may be a substituted or unsubstituted pyridinyl, pyrrolyl, imidazolyl, triazolyl, tetrazolyl, indolyl, pyridazinyl, pyrimidinyl, pyrazinyl, quinolinyl, benzimidazolyl, pyrazolyl, or azaindolyl group.

In preferred processes of the invention, X in the compound of Formula I is a substituted or unsubstituted heteroaryl group that comprises one or more oxygen atoms. For example, X may be a substituted or unsubstituted furanyl or benzofuranyl group.

In preferred processes of the invention, X in the compound of Formula I is a substituted or unsubstituted heteroaryl group that comprises one or more sulfur atoms. For example, X may be a substituted or unsubstituted thiophenyl or benzothiophenyl group.

In preferred processes of the invention, X in the compound of Formula I is a substituted or unsubstituted heteroaryl group that comprises a sulfur atom and a nitrogen atom. For example, X may be a substituted or unsubstituted thiazolyl, isothiazolyl, thiadiazolyl, or 1,2-benzthiazolyl group.

In preferred processes of the invention, X in the compound of Formula I is a substituted or unsubstituted heteroaryl group that comprises an oxygen atom and a nitrogen atom. For example, X may be a substituted or unsubstituted oxazolyl, isoxazolyl, oxadiazolyl, or benzoxazolyl group.

Thus, in preferred processes of the invention, X in the compound of Formula I is a substituted or unsubstituted heteroaryl group selected from substituted or unsubstituted pyridinyl, pyrrolyl, imidazolyl, triazolyl, tetrazolyl, indolyl, pyridazinyl, pyrimidinyl, pyrazinyl, quinolinyl, benzimidazolyl, pyrazolyl, azaindolyl, furanyl, benzofuranyl, thiophenyl, benzothiophenyl, thiazolyl, isothiazolyl, thiadiazolyl, 1,2-benzthiazolyl, oxazolyl, isoxazolyl, oxadiazolyl, and benzoxazolyl.

In a more preferred process of the invention, X in the compound of Formula I is a substituted or unsubstituted aromatic group selected from phenyl, pyridazinyl, pyrimidinyl, pyrazinyl, and pyridinyl.

It may be preferred that when X in the compound of Formula I is a substituted or unsubstituted heteroaryl group that comprises one or more nitrogen atoms, that Z is not connected to X in a position adjacent to an sp² nitrogen atom of the heteroaryl group.

In preferred processes of the invention, R in the compound of Formula I is a substituted or unsubstituted C₁₋₁₀ straight-chain alkyl group or C₃₋₁₀ branched-chain alkyl group, most preferably a substituted or unsubstituted C₁₋₄ straight-chain alkyl group or C₃₋₄ branched-chain alkyl group. In particularly preferred processes of the invention, R in the compound of Formula I is methyl, ethyl, propyl, iso-propyl, butyl, sec-butyl, or tert-butyl. Most preferably, R in the compound of Formula I is methyl.

In the compound of Formula II, Y is a substituted or unsubstituted aromatic group. In the compound of Formula II, the boron atom in —B(OR¹)(OR²) is bonded directly to an sp²-hybridised carbon atom of the substituted or unsubstituted aromatic group, Y.

In preferred processes of the invention, Y in the compound of Formula II is a substituted or unsubstituted aryl group, or a substituted or unsubstituted heteroaryl group.

In preferred processes of the invention, Y in the compound of Formula II is a substituted or unsubstituted aryl group. Preferably, Y in the compound of Formula II is a substituted or unsubstituted C₆-C₂₀ aryl group, more preferably a substituted or unsubstituted C₆-C₁₈ aryl group, even more preferably a substituted or unsubstituted C₆-C₁₀ aryl group. Preferred C₆₋₂₀-aryl groups include phenyl, tolyl, xylyl, methoxyphenyl, difluorophenyl, anthracenyl, and naphthalenyl.

In preferred processes of the invention, Y in the compound of Formula II is a substituted or unsubstituted heteroaryl group. Preferably, Y in the compound of Formula II is a substituted or unsubstituted C₄-C₂₀ heteroaryl group, more preferably a substituted or unsubstituted C₄-C₁₈ heteroaryl group, even more preferably a substituted or unsubstituted C₄-C₁₀ heteroaryl group. Preferred heteroaryl groups are discussed in more detail below.

In preferred processes of the invention, Y in the compound of Formula II is a substituted or unsubstituted heteroaryl group that comprises one, two, three, or four heteroatoms, preferably one or two heteroatoms. The heteroatom may be oxygen, nitrogen, sulfur, or phosphorous, or mixtures thereof. Typically, the heteroatom is nitrogen, sulfur, oxygen, or a mixture thereof. When Y in the compound of Formula II comprises two or more heteroatoms, the heteroatoms may be the same or different.

In preferred processes of the invention, Y in the compound of Formula II is a substituted or unsubstituted heteroaryl group that comprises one or more nitrogen atoms. For example, Y may be a substituted or unsubstituted pyridinyl, pyrrolyl, imidazolyl, triazolyl, tetrazolyl, indolyl, pyridazinyl, pyrimidinyl, pyrazinyl, quinolinyl, benzimidazolyl, pyrazolyl, or azaindolyl group.

In particularly preferred processes of the invention, Y in the compound of Formula II is a substituted or unsubstituted azaindolyl group, preferably a substituted or unsubstituted 7-azaindolyl group.

In preferred processes of the invention, Y in the compound of Formula II is a substituted or unsubstituted heteroaryl group that comprises one or more oxygen atoms. For example, Y may be a substituted or unsubstituted furanyl or benzofuranyl group.

In preferred processes of the invention, Y in the compound of Formula II is a substituted or unsubstituted heteroaryl group that comprises one or more sulfur atoms. For example, Y may be a substituted or unsubstituted thiophenyl or benzothiophenyl group.

In preferred processes of the invention, Y in the compound of Formula II is a substituted or unsubstituted heteroaryl group that comprises a sulfur atom and a nitrogen atom. For example, Y may be a substituted or unsubstituted thiazolyl, isothiazolyl, thiadiazolyl, or 1,2-benzthiazolyl group.

In preferred processes of the invention, Y in the compound of Formula II is a substituted or unsubstituted heteroaryl group that comprises an oxygen atom and a nitrogen atom. For example, Y may be a substituted or unsubstituted oxazolyl, isoxazolyl, oxadiazolyl, or benzoxazolyl group.

Thus, in preferred processes of the invention, Y in the compound of Formula II is a substituted or unsubstituted heteroaryl group selected from substituted or unsubstituted pyridinyl, pyrrolyl, imidazolyl, triazolyl, tetrazolyl, indolyl, pyridazinyl, pyrimidinyl, pyrazinyl, quinolinyl, benzimidazolyl, pyrazolyl, azaindolyl, furanyl, benzofuranyl, thiophenyl, benzothiophenyl, thiazolyl, isothiazolyl, thiadiazolyl, 1,2-benzthiazolyl, oxazolyl, isoxazolyl, oxadiazolyl, and benzoxazolyl.

In the compound of Formula II, R¹ and R² are, independently, H, or a substituted or unsubstituted organic group comprising 1-20 carbon atoms; or, together with the atoms to which they are attached, R¹ and R² form a ring.

In preferred processes of the invention, R¹ and R² in the compound of Formula II are, independently, H or a substituted or unsubstituted C₁₋₂₀ straight-chain or C₃₋₂₀ branched-chain alkyl group, more preferably H or a substituted or unsubstituted C₁₋₁₀ straight-chain or C₃₋₁₀ branched-chain alkyl group, even more preferably H or a substituted or unsubstituted C₁₋₄ straight-chain or C₃₋₄ branched-chain alkyl group. Preferably, R¹ and R² in the compound of Formula II are, independently, H, methyl, ethyl, propyl, iso-propyl, butyl, sec-butyl, or tert-butyl. Most preferably, R¹ and R² in the compound of Formula II are both H.

In alternative preferred processes of the invention, together with the atoms to which they are attached, R¹ and R² in the compound of Formula II form a ring. More preferably, R¹ and R² form a ring which is —Bpin or —Bcat. Even more preferably, R¹ and R² form a ring which is —Bpin.

In the compound of Formula III, X is as generally described above. As will be understood by a person skilled in the art, X in the compound of Formula I is necessarily the same as X in the compound of Formula III, save for the possible modification/removal of any protecting groups that may be present in the compound of Formula I under the reaction conditions.

In the compound of Formula III, Y is as generally described above. As will be understood by a person skilled in the art, Y in the compound of Formula II is necessarily the same as Y in the compound of Formula III, save for the possible modification/removal of any protecting groups that may be present in the compound of Formula II under the reaction conditions.

In the compound of Formula III, X and Y may be the same or different. Preferably, X and Y are different.

In preferred processes of the invention, the compound of Formula I has a sub-Formula Ia

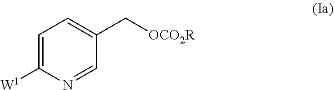

wherein R is as hereinbefore defined; and
W is a substituent selected from -halo, —C(halo)₃, —Rₐ, ═O, ═S, —O—Rₐ, —S—Rₐ, —NRₐR_{b}, —CN, —NO₂, —C(O)—Rₐ, —COORₐ, —C(S)—Rₐ, —C(S)ORₐ, —S(O)₂OH, —S(O)₂—Rₐ, —S(O)₂NRₐR_{b}, —O—S(O)—Rₐ and —CONRₐR_{b},
wherein Rₐ and R_{b} are independently selected from the groups consisting of H, substituted or unsubstituted alkyl, substituted or unsubstituted aryl, substituted or unsubstituted arylalkyl, substituted or unsubstituted heteroalkyl, substituted or unsubstituted heteroaryl, or Rₐ and R_{b} together with the atom to which they are attached form a heterocycloalkyl group.

Preferably, W¹ in sub-Formula Ia is selected from -halo, —C(halo)₃, —Rₐ, —O—Rₐ, —NRₐR_{b}, —CN, and —NO₂. More preferably, W¹ in sub-Formula Ia is —NRₐR_{b}. Even more preferably, W¹ in sub-Formula Ia is —NRₐR_{b} wherein Rₐ and R_{b} are selected from H or substituted or unsubstituted alkyl. Even more preferably, W¹ in sub-Formula Ia is —NRₐR_{b} wherein Rₐ and R_{b} are selected from H or substituted alkyl. Even more preferably, W¹ in sub-Formula Ia is —NRₐR_{b} wherein Rₐ and R_{b} are selected from H or substituted C₁-C₁₀ alkyl. Most preferably, W¹ in sub-Formula Ia is —NRₐR_{b} wherein Rₐ is H and R_{b} is substituted C₁-C₅ alkyl.

Thus, in particularly preferred processes of the invention, the compound of Formula I is

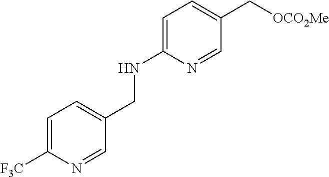

In preferred processes of the invention, the compound of Formula II has a sub-Formula IIa

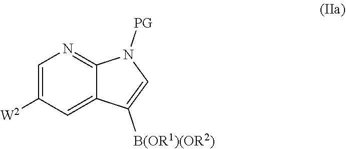

wherein R¹ and R² are as hereinbefore defined;
W² is a substituent selected from -halo, —C(halo)₃, —Rₐ, ═O, ═S, —O—Rₐ, —S—Rₐ, —NRₐR_{b}, —CN, —NO₂, —C(O)—Rₐ, —COORₐ, —C(S)—Rₐ, —C(S)ORₐ, —S(O)₂OH, —S(O)₂—Rₐ,
—S(O)₂NRₐR_{b}, —O—S(O)—Rₐ and —CONRₐR_{b},
wherein Rₐ and R_{b} are independently selected from the groups consisting of H, substituted or unsubstituted alkyl, substituted or unsubstituted aryl, substituted or unsubstituted arylalkyl, substituted or unsubstituted heteroalkyl, substituted or unsubstituted heteroaryl, or Rₐ and R_{b} together with the atom to which they are attached form a heterocycloalkyl group; and
PG is a protecting group.

Preferably, PG is selected from —H, an alkyl or aryl oxycarbonyl group (for example Boc or 9-fluorenylmethoxycarbonyl), an alkyl carbonate (for example methyl carbonate), a sulfonyl group (for example paratolyl sulfonyl), an alkyl group, an aryl group, or a silyl group (for example tri-iso-propylsilyl, tert-butyldimethylsilyl, or trimethylsilyl). More preferably, PG is Boc or —H, Most preferably, PG is Boc.

Preferably, W² in sub-Formula Ia is selected from -halo, —C(halo)₃, —Rₐ, —O—Rₐ, —NRₐR_{b}, —CN, and —NO₂. More preferably, W² in sub-Formula Ia is -halo. Even more preferably, W² in sub-Formula Ia is —Cl.

Thus, in particularly preferred processes of the invention, the compound of Formula II is

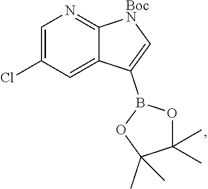 [SMILES: CC(C)(C)OC(=O)n1cc(B2OC(C)(C)C(C)(C)O2)c2cc(Cl)cnc21]

In preferred processes of the invention, the compound of Formula III has a sub-Formula IIIa

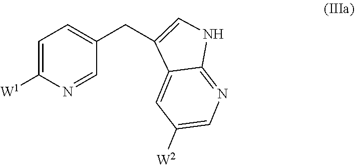

wherein W¹ and W² are as hereinbefore defined.

Accordingly, in preferred processes of the invention the compound of Formula III is

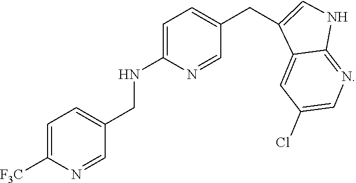 [SMILES: FC(F)(F)c1ccc(CNc2ccc(Cc3c[nH]c4ncc(Cl)cc34)cn2)cn1]

The above compound of Formula III is also known as pexidartinib. Pexidartinib is a kinase inhibitor drug for the treatment of adults with symptomatic tenosynovial giant cell tumours (TGCT). The reaction step to produce pexidartinib is depicted in FIG. 2.

The process of the invention is carried out in the presence of a catalyst. The catalyst is preferably a metal complex comprising a platinum group metal (i.e. a Group 10 metal). More preferably, the catalyst is a metal complex comprising palladium.

The catalyst may be a complex of a platinum group metal and have one or more ligands coordinated to the platinum group metal. The catalyst may be a complex of palladium and have one or more ligands coordinated to palladium.

In preferred processes of the invention, the catalyst is of formula (A)

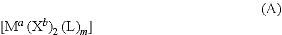

wherein,
M^{a} is a metal, preferably palladium;
X^{b} is an anionic ligand;
L is a monodentate phosphorus ligand, or a bidentate phosphorus ligand;
m is 1 or 2, wherein,
when m is 1, L is a bidentate phosphorus ligand; and
when m is 2, each L is a monodentate phosphorus ligand.

In preferred catalysts of formula (A), X^{b} is a halo group, such as —Cl, —Br, and —I, or trifluoroacetate (i.e. F₃CCO₂⁻). Preferably, X^{b} is —Cl.

In the catalysts of formula (A), L is a monodentate phosphorus ligand, or a bidentate phosphorus ligand. Any suitable phosphorus compound capable of forming a ligand-metal interaction with the M atom may be used. In the ligand, each phosphorus atom is covalently bonded to either 3 carbon atoms (tertiary phosphines) or to x heteroatoms and 3−x carbon atoms, where x=1, 2 or 3. Preferably, the heteroatom is selected from the group consisting of N and O.

The phosphorus ligand L may be monodentate, e.g. PPh₃, or bidentate.

The phosphorous ligand L may be chiral or achiral.

The phosphorous ligand L may be substituted or unsubstituted.

Phosphorus ligands L that may be used in the invention include but are not restricted to the following structural types:

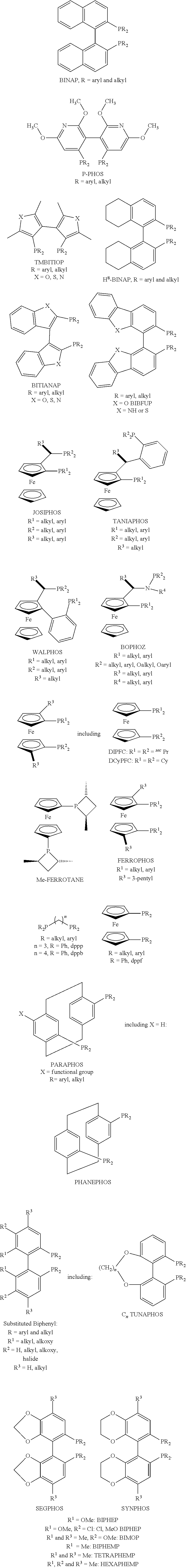

In the above structures —PR₂ may be —P(alkyl)₂ in which alkyl is preferably C₁-C₁₀ alkyl, —P(aryl)₂ where aryl includes phenyl and naphthyl which may be substituted or unsubstituted or —P(O-alkyl)₂ and —P(O-aryl)₂ with alkyl and aryl as defined above. —PR₂ may also be substituted or unsubstituted —P(heteroaryl)₂, where heteroaryl includes furanyl (e.g. 2-furanyl or 3-furanyl). —PR₂ is preferably either —P(aryl)₂ where aryl includes phenyl, tolyl, xylyl or anisyl or —P(O-aryl)₂. If —PR₂ is —P(O-aryl)₂, the most preferred O-aryl groups are those based on chiral or achiral substituted 1,1′-biphenol and 1,1′-binapthol. Alternatively, the R groups on the P-atom may be linked as part of a cyclic structure.

Substituting groups may be present on the alkyl or aryl substituents in the phosphorus ligands. Such substituting groups are typically branched or linear C₁₋₆ alkyl groups such as methyl, ethyl, propyl, iso-propyl, and tert-butyl.

These phosphorus ligands depicted above are generally available commercially and their preparation is known.

Preferred bidentate phosphorus ligands L include Binap ligands, PPhos ligands, PhanePhos ligands, Josiphos ligands and Bophoz ligands, preferably Binap ligands.

Preferred bidentate phosphorus ligands L are also ligands of formula R⁶R⁷P(CH₂)ₙPR⁸R⁹, wherein n is an integer selected from 1 to 10, preferably 2 to 6 (e.g. 3 or 4) and R⁶, R⁷, R⁸, and R⁹ may be independently selected from the group consisting of unsubstituted C₁₋₂₀-alkyl, substituted C₁₋₂₀-alkyl, unsubstituted substituted C₃₋₂₀-cycloalkyl, C₃₋₂₀-cycloalkyl, unsubstituted C₁₋₂₀-alkoxy, substituted C₁₋₂₀-alkoxy, unsubstituted C₆₋₂₀-aryl, substituted C₆₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl. R⁶, R⁷, R⁸, and R⁹ may be independently substituted or unsubstituted branched- or straight-chain alkyl groups such as methyl, ethyl, n-propyl, iso-propyl, n-butyl, iso-butyl, sec-butyl, tert-butyl, pentyl, hexyl, heptyl, octyl, nonyl, decyl, dodecyl or stearyl, cycloalkyl groups such as cyclopropyl, cyclobutyl, cyclopentyl, cyclohexyl or adamantyl, or aryl groups such as phenyl, naphthyl or anthracyl. In one embodiment, the alkyl groups may be optionally substituted with one or more substituents such as halide (F, Cl, Br or I) or alkoxy groups, e.g. methoxy, ethoxy or propoxy. The aryl group may be optionally substituted with one or more (e.g. 1, 2, 3, 4, or 5) substituents such as halide (—F, —Cl, —Br or —I), straight- or branched-chain C₁-C₁₀-alkyl (e.g. methyl), C₁-C₁₀ alkoxy, straight- or branched-chain C₁-C₁₀-(dialkyl)amino, C₃₋₁₀ heterocycloalkyl groups (such as morpholinyl and piperadinyl) or tri(halo)methyl (e.g. F₃C—). Substituted or unsubstituted heteroaryl groups such as pyridyl may also be used. In an alternative embodiment, R⁶ and R⁷ and/or R⁸ and R⁹ may be linked to form a ring structure with the phosphorus atom, preferably 4- to 7-membered rings. Preferably, the bidentate phosphorous ligand is selected from dppm (1,3-bis(diphenylphosphino)methane), dppe (1,3-bis(diphenylphosphino)ethane), dppp (1,3-bis(diphenylphosphino)propane), dppb (1,4-bis(diphenylphosphino)butane), 1,3-bis(diphenylphosphino)pentane, and 1,3-bis(diphenylphosphino)hexane, more preferably dppp and dppb.

Preferred bidentate phosphorus ligands L are also ligands of formula (A1)

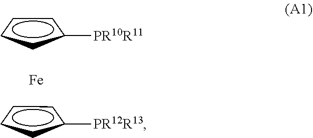

wherein R¹⁰, R¹¹, R¹², and R¹³ may be independently selected from the group consisting of unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₆₋₂₀-aryl, substituted C₆₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl. R¹⁰, R¹¹, R¹², and R¹³ may be independently substituted or unsubstituted branched- or straight-chain alkyl groups such as methyl, ethyl, n-propyl, iso-propyl, n-butyl, iso-butyl, sec-butyl, tert-butyl, pentyl, hexyl, heptyl, octyl, nonyl, decyl, dodecyl or stearyl, cycloalkyl groups such as cyclopropyl, cyclobutyl, cyclopentyl, cyclohexyl or adamantyl, or aryl groups such as phenyl, naphthyl or anthracyl. In one embodiment, the alkyl groups may be optionally substituted with one or more substituents such as halide (F, Cl, Br or I) or alkoxy groups, e.g. methoxy, ethoxy or propoxy. The aryl group may be optionally substituted with one or more (e.g. 1, 2, 3, 4, or 5) substituents such as halide (—F, —Cl, —Br or —I), straight- or branched-chain C₁-C₁₀-alkyl (e.g. methyl), C₁-C₁₀ alkoxy, straight- or branched-chain C₁-C₁₀-(dialkyl)amino, C₃₋₁₀ heterocycloalkyl groups (such as morpholinyl and piperadinyl) or tri(halo)methyl (e.g. F₃C—). Substituted or unsubstituted heteroaryl groups such as pyridyl may also be used. Preferably, the bidentate ligand is selected from dppf (1,1′-bis(diphenylphosphino)ferrocene), dippf (1,1′-bis(di-isopropylphosphino)ferrocene), dCyPfc (1,1′-bis(di-cyclohexylphosphino)ferrocene and dtbpf (1,1′-bis(di-tert-butylphosphino)ferrocene), more preferably dippf and dCyPfc.

Preferred monodentate phosphorous ligands L are tertiary phosphine ligands of the formula PR¹⁴R¹⁵R¹⁶. R¹⁴, R¹⁵ and R¹⁶ may be independently selected from the group consisting of unsubstituted C₁₋₂₀-alkyl, substituted C₁₋₂₀-alkyl, unsubstituted C₃₋₂₀-cycloalkyl, substituted C₃₋₂₀-cycloalkyl, unsubstituted C₁₋₂₀-alkoxy, substituted C₁₋₂₀-alkoxy, unsubstituted C₆₋₂₀-aryl, substituted C₆₋₂₀-aryl, unsubstituted C₁₋₂₀-heteroalkyl, substituted C₁₋₂₀-heteroalkyl, unsubstituted C₂₋₂₀-heterocycloalkyl, substituted C₂₋₂₀-heterocycloalkyl, unsubstituted C₄₋₂₀-heteroaryl and substituted C₄₋₂₀-heteroaryl. R¹⁴, R¹⁵ and R¹⁶ may be independently substituted or unsubstituted branched- or straight-chain alkyl groups such as methyl, ethyl, n-propyl, iso-propyl, n-butyl, iso-butyl, sec-butyl, tert-butyl, pentyl, hexyl, heptyl, octyl, nonyl, decyl, dodecyl or stearyl, cycloalkyl groups such as cyclopropyl, cyclobutyl, cyclopentyl, cyclohexyl or adamantyl, or aryl groups such as phenyl, naphthyl or anthracyl. In one embodiment, the alkyl groups may be optionally substituted with one or more substituents such as halide (F, Cl, Br or I) or alkoxy groups, e.g. methoxy, ethoxy or propoxy. The aryl group may be optionally substituted with one or more (e.g. 1, 2, 3, 4, or 5) substituents such as halide (—F, —Cl, —Br or —I), straight- or branched-chain C₁-C₁₀-alkyl (e.g. methyl), C₁-C₁₀ alkoxy, straight- or branched-chain C₁-C₁₀-(dialkyl)amino, C₃₋₁₀ heterocycloalkyl groups (such as morpholinyl and piperadinyl) or tri(halo)methyl (e.g. F₃C—). Substituted or unsubstituted heteroaryl groups such as pyridyl may also be used. In an alternative embodiment, any two of R¹⁴, R¹⁵ and R¹⁶ may be linked to form a ring structure with the phosphorus atom, preferably 4- to 7-membered rings. Preferably, R¹⁴, R¹⁵ and R¹⁶ are the same and are phenyl, i.e. PR¹⁴R¹⁵R¹⁶ is triphenylphosphine. Alternatively, R¹⁴, R¹⁵ and R¹⁶ may be the same and are tolyl, i.e. PR¹⁴R¹⁵R¹⁶ is tritolylphosphine (e.g. ortho-, meta- or para- tritolylphosphine). Alternatively, R¹⁴, R¹⁵ and R¹⁶ are the same and are cyclohexyl, i.e. PR¹⁴R¹⁵R¹⁶ is tricyclohexylphosphine. Alternatively, R¹⁴, R¹⁵ and R¹⁶ are the same and are tert-butyl, i.e. PR¹⁴R¹⁵R¹⁶ is tri(tert-butyl)phosphine.

Preferred phosphorus ligands L are selected from the group consisting of PPh₃, tritolylphosphine, PCy₃ (tricyclohexylphosphine), P^{t}Bu₃, dppm (1,3-bis(diphenylphosphino)methane), dppe (1,3-bis(diphenylphosphino)ethane), dppp (1,3-bis(diphenylphosphino)propane), dppb (1,4-bis(diphenylphosphino)butane), 1,3-bis(diphenylphosphino)pentane, 1,3-bis(diphenylphosphino)hexane, dppf (1,1′-bis(diphenylphosphino)ferrocene), dippf (1,1′-bis(di-isopropylphosphino)ferrocene), dCyPfc (1,1′-bis(di-cyclohexylphosphino)ferrocene and dtbpf (1,1′-bis(di-tert-butylphosphino)ferrocene).

Particularly preferred phosphorus ligands L are selected from the group consisting of dppp, dppb, dppf, dippf, dtbpf and dCyPfc, more preferably dippf, dtbpf and dCyPfc, even more preferably dippf.

In preferred processes of the invention, the catalyst is selected from PdX^{b}₂(dppp), PdX^{b}₂(dppb), PdX^{b}₂(dppf), PdX^{b}₂(dippf), PdX^{b}₂(dtbpf) and PdX^{b}₂(dCyPfc) wherein X^{b} is as defined above, more preferably PdX^{b}₂(dippf), PdX^{b}₂(dtbpf) and PdX^{b}₂(dCyPfc), even more preferably PdX^{b}₂(dippf).

In preferred processes of the invention, the catalyst is selected from PdCl₂(dppp), PdCl₂(dppb), PdCl₂(dppf), PdCl₂(dippf), PdCl₂(dtbpf) and PdCl₂(dCyPfc), more preferably PdCl₂(dippf), PdCl₂(dtbpf) and PdCl₂(dCyPfc), even more preferably PdCl₂(dippf).

In alternative preferred processes of the invention, the catalyst is of formula (B)

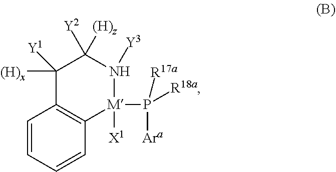

wherein,
M′ is a metal, preferably palladium;
X¹ is an anionic ligand;
Ar^{a} is a substituted or unsubstituted aryl group, a substituted or unsubstituted heteroaryl group, or a substituted or unsubstituted xanthenyl group;
R^{17a} and R^{18b} are independently organic groups having 1-20 carbon atoms, or R^{17a} and R^{18a} are linked to form a ring structure with P;
each of Y¹ and Y² is hydrogen; or together with the atoms to which they are attached, Y¹ and Y² form an aromatic ring;
Y³ is hydrogen, a substituted or unsubstituted C₁₋₂₀ straight-chain or C₃₋₂₀ branched-chain alkyl group or a substituted or unsubstituted C₆₋₂₀ aryl group;
x and z are 0 or 1, wherein
when x and z are both 1, each of Y¹ and Y² is hydrogen; and
when x and z are both 0, together with the atoms to which they are attached, Y¹ and Y² form an aromatic ring.

In the catalysts of formula (B), X¹ is an anionic ligand. X¹ may be a coordinated anionic ligand or a non-coordinated anionic ligand.

In the catalysts of formula (B), X¹ is preferably a halo group, such as —Cl, —Br, and —I, or mesylate (i.e. MsO⁻ or MeSO₃⁻). More preferably, X¹ is —Cl.

In preferred catalysts of formula (B), each of Y¹ and Y² is hydrogen. In this instance, x and z are both 1.

In alternative preferred catalysts of formula (B), together with the atoms to which they are attached, Y¹ and Y² form an aromatic ring. In this instance, x and z are both 0. Preferably, the aromatic ring is a six-membered aromatic ring. More preferably, together with the atoms to which they are attached, Y¹ and Y² form a benzene ring.

In the catalysts of formula (B), Y³ is preferably hydrogen, a substituted or unsubstituted C₁₋₁₀ straight-chain or C₃₋₁₀ branched-chain alkyl group or a substituted or unsubstituted C₆₋₁₀ aryl group. More preferably, Y³ is hydrogen, a substituted or unsubstituted C₁₋₅ straight-chain or C₃₋₅ branched-chain alkyl group or a substituted or unsubstituted C₆₋₁₀ aryl group. Most preferably, Y³ is hydrogen, methyl or phenyl.

In the catalysts of formula (B), R^{17a} and R^{18a} may be the same or different. In one embodiment, R^{17a} and R^{18a} are the same. In another embodiment, R^{17a} and R^{18a} are different. R^{17a} and R^{18a} are selected up to the limitations imposed by stability and the rules of valence. R^{17a} and R^{18a} may be independently selected from the group consisting of substituted and unsubstituted straight-chain C₁₋₂₀-alkyl, substituted and unsubstituted branched-chain C₃₋₂₀-alkyl, substituted and unsubstituted C₃₋₂₀-cycloalkyl, substituted and unsubstituted C₆₋₂₀-aryl, and substituted and unsubstituted C₄₋₂₀-heteroaryl wherein the heteroatoms are independently selected from sulfur, nitrogen and oxygen. R^{17a} and R^{18a} may independently be substituted or unsubstituted branched- or straight-chain alkyl groups such as methyl, ethyl, n-propyl, iso-propyl, n-butyl, iso-butyl, sec-butyl, tert-butyl, pentyl (e.g. n-pentyl or neopentyl), hexyl, heptyl, octyl, nonyl, decyl, dodecyl or stearyl, cycloalkyl groups such as cyclopropyl, cyclobutyl, cyclopentyl, cyclohexyl or adamantyl, or aryl groups such as phenyl, naphthyl or anthracyl. In one embodiment, the alkyl groups may be optionally substituted with one or more (e.g. 1, 2, 3, 4, or 5) substituents each of which may be the same or different such as halide (F, Cl, Br or I) or alkoxy groups, e.g. methoxy, ethoxy or propoxy. The aryl group may be optionally substituted with one or more (e.g. 1, 2, 3, 4, or 5) substituents each of which may be the same or different such as halide (F, Cl, Br or I), straight- or branched-chain alkyl (e.g. C₁-C₁₀), alkoxy (e.g. C₁-C₁₀ alkoxy), straight- or branched-chain (dialkyl)amino (e.g. C₁-C₁₀ dialkyl)amino), heterocycloalkyl (e.g. C₃₋₁₀ heterocycloalkyl groups, such as morpholinyl and piperadinyl) or tri(halo)methyl (e.g. F₃C—). Suitable substituted aryl groups include but are not limited to 4-dimethylaminophenyl, 4-methylphenyl, 3,5-dimethylphenyl, 4-methoxyphenyl, 4-methoxy-3,5-dimethylphenyl and 3,5-di(trifluoromethyl)phenyl. Substituted or unsubstituted heteroaryl groups such as pyridyl may also be used. In an alternative embodiment, R^{17a} and R^{18a} are linked to form a ring structure with P, preferably 4- to 7-membered rings. Preferably, R^{17a} and R^{18a} are the same and are tert-butyl, cyclohexyl, adamantyl, phenyl or substituted phenyl groups, such as 3,5-di(trifluoromethyl)phenyl.

In the catalysts of formula (B), Ar^{a} is a substituted or unsubstituted aryl group, a substituted or unsubstituted heteroaryl group, or a substituted or unsubstituted xanthenyl group. Preferably, Ar^{a} is a substituted or unsubstituted phenyl group, a substituted or unsubstituted biphenyl group, a substituted or unsubstituted napthyl group, a substituted or unsubstituted binapthyl group, a substituted or unsubstituted pyrazolyl group, a substituted or unsubstituted bipyrazolyl group, or a substituted or unsubstituted xanthenyl group. More preferably, Ar^{a} is a substituted or unsubstituted biphenyl group, a substituted or unsubstituted binapthyl group, a substituted or unsubstituted bipyrazolyl group, or a substituted or unsubstituted xanthenyl group. Even more preferably, Ar^{a} is a substituted or unsubstituted biphenyl group, a substituted or unsubstituted bipyrazolyl group, or a substituted or unsubstituted xanthenyl group.

In the catalysts of formula (B), the phosphine ligand —P(R^{17a})(R^{18b})Ar^{a} is preferably selected from the group consisting of:

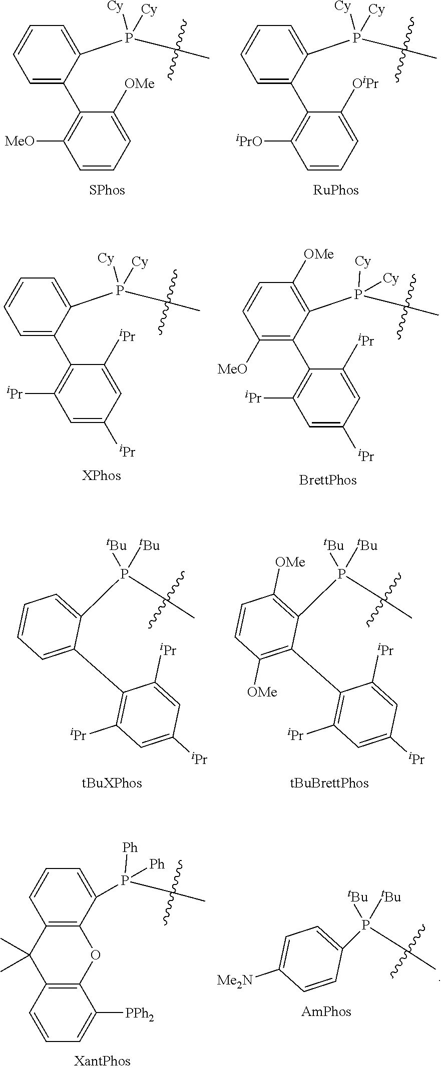

In preferred processes of the invention, the catalyst is selected from:

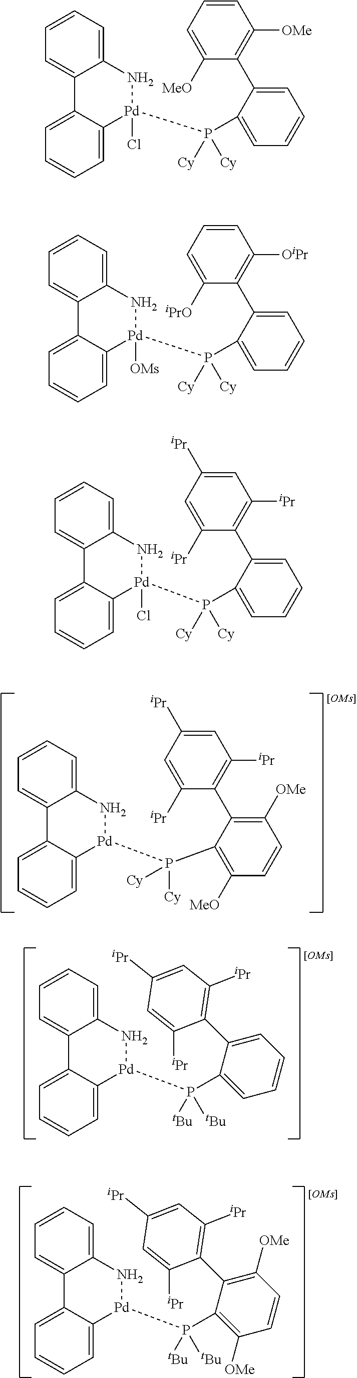

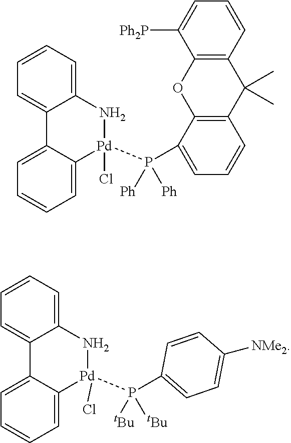

In alternative preferred processes of the invention, the catalyst is of formula (Ca)

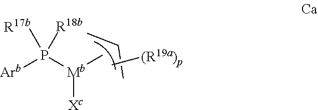

wherein,
M^{b} is a metal, preferably palladium;
X^{c} is a coordinated anionic ligand;
Ar^{b} is a substituted or unsubstituted aryl group or a substituted or unsubstituted heteroaryl group;
R^{17b} and R^{18b} are independently organic groups having 1-20 carbon atoms, or R^{17b} and R^{18b} are linked to form a ring structure with P;
R^{19a} is an organic group having 1-20 carbon atoms; and
p is 0, 1, 2, 3, 4 or 5.

In the catalysts of formula (Ca), X^{c} is a coordinated anionic ligand i.e. the anionic ligand is bonded to the Pd atom within the coordination sphere. X^{c} is preferably a halo group, such as —Cl, —Br, and —I, or trifluoroacetate (i.e. F₃CCO₂⁻). More preferably, X^{c} is —Cl.

In the catalysts of formula (Ca), R^{17b} and R^{18b} may be the same or different. In one embodiment, R^{17b} and R^{18b} are the same. In another embodiment, R^{17b} and R^{18b} are different. R^{17b} and R^{18b} are selected up to the limitations imposed by stability and the rules of valence. R^{17b} and R^{18b} may be independently selected from the group consisting of substituted and unsubstituted straight-chain C₁₋₂₀-alkyl, substituted and unsubstituted branched-chain C₃₋₂₀-alkyl, substituted and unsubstituted C₃₋₂₀-cycloalkyl, substituted and unsubstituted C₆₋₂₀-aryl, and substituted and unsubstituted C₄₋₂₀-heteroaryl wherein the heteroatoms are independently selected from sulfur, nitrogen and oxygen. R^{17b} and R^{18b} may independently be substituted or unsubstituted branched- or straight-chain alkyl groups such as methyl, ethyl, n-propyl, iso-propyl, n-butyl, iso-butyl, sec-butyl, tert-butyl, pentyl (e.g. n-pentyl or neopentyl), hexyl, heptyl, octyl, nonyl, decyl, dodecyl or stearyl, cycloalkyl groups such as cyclopropyl, cyclobutyl, cyclopentyl, cyclohexyl or adamantyl, or aryl groups such as phenyl, naphthyl or anthracyl. In one embodiment, the alkyl groups may be optionally substituted with one or more (e.g. 1, 2, 3, 4, or 5) substituents each of which may be the same or different such as halide (F, Cl, Br or I) or alkoxy groups, e.g. methoxy, ethoxy or propoxy. The aryl group may be optionally substituted with one or more (e.g. 1, 2, 3, 4, or 5) substituents each of which may be the same or different such as halide (F, Cl, Br or I), straight- or branched-chain alkyl (e.g. C₁-C₁₀), alkoxy (e.g. C₁-C₁₀ alkoxy), straight- or branched-chain (dialkyl)amino (e.g. C₁-C₁₀ dialkyl)amino), heterocycloalkyl (e.g. C₃₋₁₀ heterocycloalkyl groups, such as morpholinyl and piperadinyl) or tri(halo)methyl (e.g. F₃C—). Suitable substituted aryl groups include but are not limited to 4-dimethylaminophenyl, 4-methylphenyl, 3,5-dimethylphenyl, 4-methoxyphenyl, 4-methoxy-3,5-dimethylphenyl and 3,5-di(trifluoromethyl)phenyl. Substituted or unsubstituted heteroaryl groups such as pyridyl may also be used. In an alternative embodiment, R^{17b} and R^{18b} are linked to form a ring structure with P, preferably 4- to 7-membered rings. Preferably, R^{17b} and R^{18b} are the same and are tert-butyl, cyclohexyl, phenyl or substituted phenyl groups, such as 3,5-di(trifluoromethyl)phenyl. Alternatively, R^{17b} and R^{18b} are independently selected from the group consisting of -Me, -Et, —ⁿPr, —ⁱPr, -ⁿBu, -ⁱBu, cyclohexyl and cycloheptyl.

In the catalysts of formula (Ca), Ar^{b} is a substituted or unsubstituted aryl group, or a substituted or unsubstituted heteroaryl group. Preferably, Ar^{b} is a substituted or unsubstituted phenyl group, a substituted or unsubstituted biphenyl group, a substituted or unsubstituted napthyl group, a substituted or unsubstituted binapthyl group, a substituted or unsubstituted pyrazolyl group, or a substituted or unsubstituted bipyrazolyl group. More preferably, Ar^{b} is a substituted or unsubstituted biphenyl group, a substituted or unsubstituted binapthyl group, or a substituted or unsubstituted bipyrazolyl group. Even more preferably, Ar^{b} is a substituted or unsubstituted biphenyl group, or a substituted or unsubstituted bipyrazolyl group.

In the catalysts of formula (Ca), the phosphine ligand —P(R^{17b})(R^{18b})Ar^{b} is preferably selected from the group consisting of:

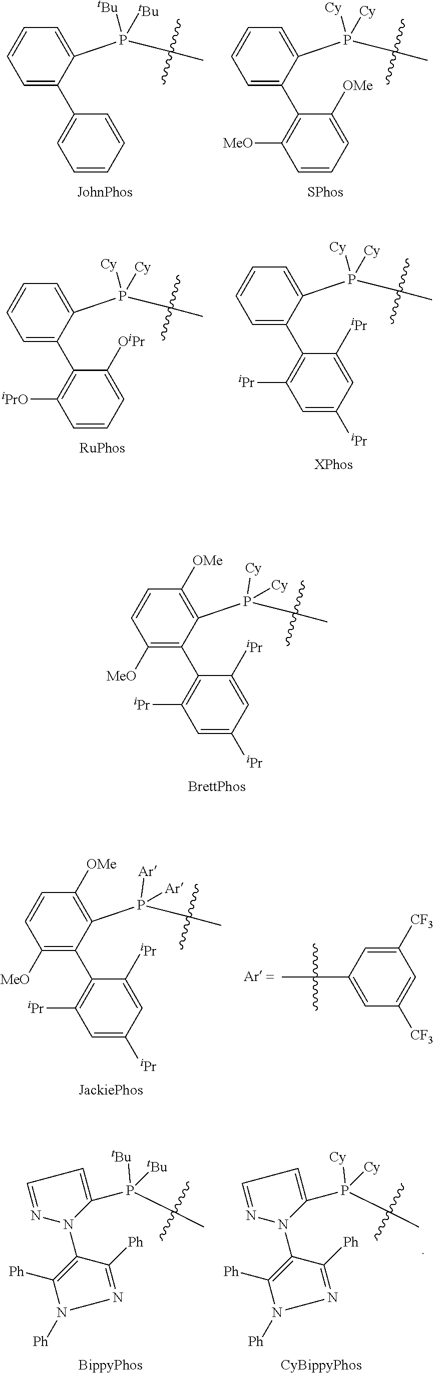

The metal atom in the catalysts of formula (Ca) is coordinated to an optionally substituted allyl group. R^{19a} is an organic group having 1-20 carbon atoms, preferably 1-10 carbon atoms and more preferably 1-8 carbon atoms. R^{19a} is selected up to the limitations imposed by stability and the rules of valence. The number of R^{19a} groups ranges from 0 to 5 i.e. p is 0, 1, 2, 3, 4 or 5. When p is 2, 3, 4 or 5, each of R^{19a} may be the same or different. In certain embodiments, when p is 2, 3, 4, or 5, each R^{19a} is the same. In certain embodiments, p is 0 (i.e. the allyl group is unsubstituted). In certain embodiments, p is 1. In certain embodiments, p is 2, wherein each R^{19a} is the same or different.

In the catalysts of formula (Ca), R^{19a} may be selected from the group consisting of substituted and unsubstituted straight-chain C₁₋₂₀-alkyl, substituted and unsubstituted branched-chain C₃₋₂₀-alkyl, substituted and unsubstituted C₃₋₂₀-cycloalkyl, substituted and unsubstituted C₆₋₂₀-aryl, and substituted and unsubstituted C₄₋₂₀-heteroaryl wherein the heteroatoms are independently selected from sulfur, nitrogen and oxygen. In one embodiment, R^{19a} is selected from the group consisting of substituted and unsubstituted straight-chain C₁₋₂₀-alkyl, substituted and unsubstituted branched-chain C₃₋₂₀-alkyl, and substituted and unsubstituted C₃₋₂₀-cycloalkyl. In another embodiment, R^{19a} is selected from the group consisting of substituted and unsubstituted C₆₋₂₀-aryl, and substituted and unsubstituted C₄₋₂₀-heteroaryl wherein the heteroatoms are independently selected from sulfur, nitrogen and oxygen. R^{19a} may be substituted or unsubstituted branched- or straight-chain alkyl groups such as methyl, ethyl, n-propyl, iso-propyl, n-butyl, iso-butyl, sec-butyl, tert-butyl, pentyl, hexyl, heptyl, octyl, nonyl, decyl, dodecyl or stearyl, cycloalkyl groups such as cyclopropyl, cyclobutyl, cyclopentyl, cyclohexyl or adamantyl or aryl groups such as phenyl, naphthyl or anthracyl. In one embodiment, the alkyl groups may be optionally substituted with one or more (e.g. 1, 2, 3, 4, or 5) substituents each of which may be the same or different such as halide (F, Cl, Br or I), alkoxy groups, e.g. methoxy, ethoxy or propoxy. The aryl group may be optionally substituted with one or more (e.g. 1, 2, 3, 4, or 5) substituents each of which may be the same or different such as halide (F, Cl, Br or I), straight- or branched-chain alkyl (e.g. C₁-C₁₀), alkoxy (e.g. C₁-C₁₀ aloxy), straight- or branched-chain (dialkyl)amino (e.g. C₁-C₁₀ dialkyl)amino), heterocycloalkyl (e.g. C₃₋₁₀ heterocycloalkyl groups, such as morpholinyl and piperadinyl) or tri(halo)methyl (e.g. F₃C—). Suitable substituted aryl groups include but are not limited to 2-, 3- or 4-dimethylaminophenyl, 2-, 3- or 4-methylphenyl, 2,3- or 3,5-dimethylphenyl, 2-, 3- or 4-methoxyphenyl and 4-methoxy-3,5-dimethylphenyl. Substituted or unsubstituted heteroaryl groups such as pyridyl may also be used. In one embodiment, each R¹⁸ is independently a methyl, phenyl or substituted phenyl group.

In preferred processes of the invention, the catalyst is selected from

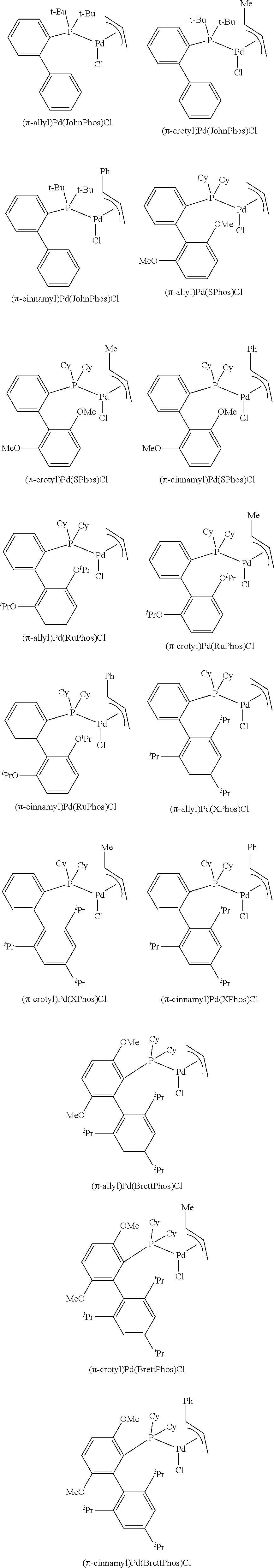

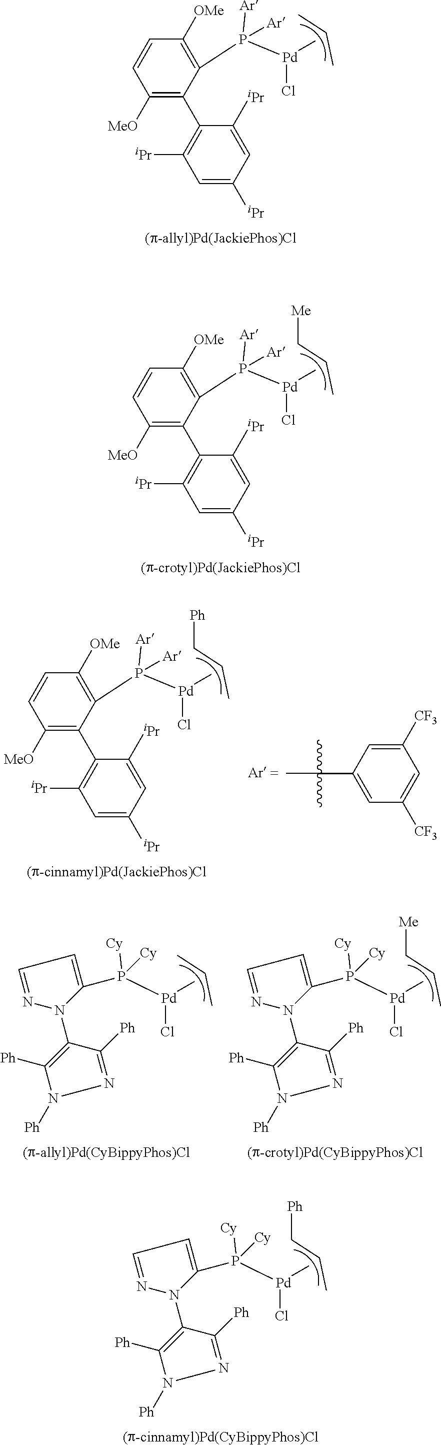

In alternative preferred processes of the invention, the catalyst is of formula (Cb)

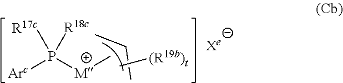

wherein,
M″^{⊕} is a cationic metal atom, preferably a cationic palladium atom;
X^{e ⊖} is a non-coordinated anionic ligand;
Ar^{c} is a substituted or unsubstituted aryl group or a substituted or unsubstituted heteroaryl group;
R^{17c} and R^{18c} are independently organic groups having 1-20 carbon atoms, or R^{17c} and R^{18c} are linked to form a ring structure with P;
R^{19b} is an organic group having 1-20 carbon atoms; and
t is 0, 1, 2, 3, 4 or 5.

In the catalysts of formula (Cb), X^{e⊖} is a non-coordinated anionic ligand. By “non-coordinated anion ligand”, we mean the anionic ligand is forced to the outer sphere of the metal centre. The anionic ligand, therefore, is dissociated from the metal centre. This is in contrast to neutral complexes in which the anionic ligand is bound to the metal within the coordination sphere. The anionic ligand can be generally identified as non-coordinating by analysing the X-ray crystal structure of the cationic complex. In one embodiment, X^{e⊖} is selected from the group consisting of triflate (i.e. TfO⁻ or CF₃SO₃⁻), tetrafluoroborate (i.e. ⁻BF₄), hexafluoroantimonate (i.e. ⁻SbF₆), hexafluorophosphate (PF₆⁻), [B[3,5-(CF₃)₂C₆H₃]₄]⁻ ([Bar^{F}₄]⁻) and mesylate (MsO⁻ or MeSO₃⁻).

In the catalysts of formula (Cb), R^{17c} and R^{18c} may be the same or different. In one embodiment, R^{17c} and R^{18c} are the same. In another embodiment, R^{17c} and R^{18c} are different. R^{17c} and R^{18c} are selected up to the limitations imposed by stability and the rules of valence. R^{17c} and R^{18c} may be independently selected from the group consisting of substituted and unsubstituted straight-chain C₁₋₂₀-alkyl, substituted and unsubstituted branched-chain C₃₋₂₀-alkyl, substituted and unsubstituted C₃₋₂₀-cycloalkyl, substituted and unsubstituted C₆₋₂₀-aryl, and substituted and unsubstituted C₄₋₂₀-heteroaryl wherein the heteroatoms are independently selected from sulfur, nitrogen and oxygen. R^{17c} and R^{18c} may independently be substituted or unsubstituted branched- or straight-chain alkyl groups such as methyl, ethyl, n-propyl, iso-propyl, n-butyl, iso-butyl, sec-butyl, tert-butyl, pentyl (e.g. n-pentyl or neopentyl), hexyl, heptyl, octyl, nonyl, decyl, dodecyl or stearyl, cycloalkyl groups such as cyclopropyl, cyclobutyl, cyclopentyl, cyclohexyl or adamantyl, or aryl groups such as phenyl, naphthyl or anthracyl. In one embodiment, the alkyl groups may be optionally substituted with one or more (e.g. 1, 2, 3, 4, or 5) substituents each of which may be the same or different such as halide (F, Cl, Br or I) or alkoxy groups, e.g. methoxy, ethoxy or propoxy. The aryl group may be optionally substituted with one or more (e.g. 1, 2, 3, 4, or 5) substituents each of which may be the same or different such as halide (F, Cl, Br or I), straight- or branched-chain alkyl (e.g. C₁-C₁₀), alkoxy (e.g. C₁-C₁₀ alkoxy), straight- or branched-chain (dialkyl)amino (e.g. C₁-C₁₀ dialkyl)amino), heterocycloalkyl (e.g. C₃₋₁₀ heterocycloalkyl groups, such as morpholinyl and piperadinyl) or tri(halo)methyl (e.g. F₃C—). Suitable substituted aryl groups include but are not limited to 4-dimethylaminophenyl, 4-methylphenyl, 3,5-dimethylphenyl, 4-methoxyphenyl, 4-methoxy-3,5-dimethylphenyl and 3,5-di(trifluoromethyl)phenyl. Substituted or unsubstituted heteroaryl groups such as pyridyl may also be used. In an alternative embodiment, R^{17c} and R^{18c} are linked to form a ring structure with P, preferably 4- to 7-membered rings. Preferably, R^{17c} and R^{18c} are the same and are tert-butyl, cyclohexyl, adamantyl, phenyl or substituted phenyl groups, such as 3,5-di(trifluoromethyl)phenyl.

In the catalysts of formula (Cb), Ar^{c} is a substituted or unsubstituted aryl group, or a substituted or unsubstituted heteroaryl group. Preferably, Ar^{c} is a substituted or unsubstituted phenyl group, a substituted or unsubstituted biphenyl group, a substituted or unsubstituted napthyl group, a substituted or unsubstituted binapthyl group, a substituted or unsubstituted pyrazolyl group, or a substituted or unsubstituted bipyrazolyl group. More preferably, Ar^{c} is a substituted or unsubstituted biphenyl group, a substituted or unsubstituted binapthyl group, or a substituted or unsubstituted bipyrazolyl group. Even more preferably, Ar^{c} is a substituted or unsubstituted biphenyl group, or a substituted or unsubstituted bipyrazolyl group.

In the catalysts of formula (Cb), the phosphine ligand —P(R^{17c})(R^{18c})Ar^{c} is preferably selected from the group consisting of:

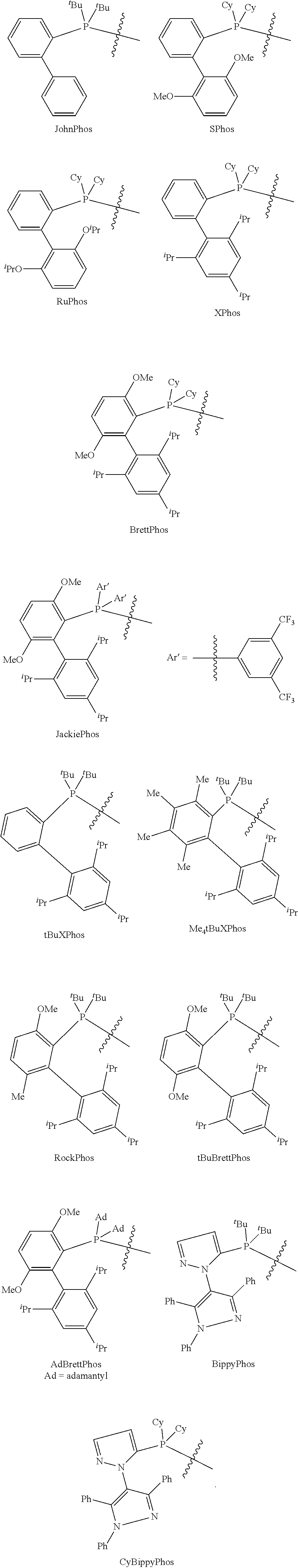

The metal cation in the catalysts of formula (Cb) is coordinated to an optionally substituted allyl group. R^{19b} is an organic group having 1-20 carbon atoms, preferably 1-10 carbon atoms and more preferably 1-8 carbon atoms. R^{19b} is selected up to the limitations imposed by stability and the rules of valence. The number of R^{19b} groups ranges from 0 to 5 i.e. t is 0, 1, 2, 3, 4 or 5. When t is 2, 3, 4 or 5, each of R^{19b} may be the same or different. In certain embodiments, when t is 2, 3, 4, or 5, each R^{19b} is the same. In certain embodiments, t is 0 i.e. the allyl group is unsubstituted. In certain embodiments, t is 1. In certain embodiments, t is 2, wherein each R^{19b} is the same or different.

In the catalysts of formula (Ca), R^{19b} may be selected from the group consisting of substituted and unsubstituted straight-chain alkyl, substituted and unsubstituted branched-chain alkyl, substituted and unsubstituted cycloalkyl, substituted and unsubstituted aryl, and substituted and unsubstituted heteroaryl wherein the heteroatoms are independently selected from sulfur, nitrogen and oxygen. In one embodiment, R^{19b} is selected from the group consisting of substituted and unsubstituted straight-chain alkyl, substituted and unsubstituted branched-chain alkyl, and substituted and unsubstituted cycloalkyl. In another embodiment, R^{19b} is selected from the group consisting of substituted and unsubstituted aryl, and substituted and unsubstituted heteroaryl wherein the heteroatoms are independently selected from sulfur, nitrogen and oxygen. R^{19b} may be substituted or unsubstituted branched- or straight-chain alkyl groups such as methyl, ethyl, n-propyl, iso-propyl, n-butyl, iso-butyl, sec-butyl, tert-butyl, pentyl, hexyl, heptyl, octyl, nonyl, decyl, dodecyl or stearyl, cycloalkyl groups such as cyclopropyl, cyclobutyl, cyclopentyl, cyclohexyl or adamantyl or aryl groups such as phenyl, naphthyl or anthracyl. In one embodiment, the alkyl groups may be optionally substituted with one or more (e.g. 1, 2, 3, 4, or 5) substituents each of which may be the same or different such as halide (F, Cl, Br or I), alkoxy groups, e.g. methoxy, ethoxy or propoxy. The aryl group may be optionally substituted with one or more (e.g. 1, 2, 3, 4, or 5) substituents each of which may be the same or different such as halide (F, Cl, Br or I), straight- or branched-chain alkyl (e.g. C₁-C₁₀), alkoxy (e.g. C₁-C₁₀ alkoxy), straight- or branched-chain (dialkyl)amino (e.g. C₁-C₁₀ dialkyl)amino), heterocycloalkyl (e.g. C₃₋₁₀ heterocycloalkyl groups, such as morpholinyl and piperadinyl) or tri(halo)methyl (e.g. F₃C—). Suitable substituted aryl groups include but are not limited to 2-, 3- or 4-dimethylaminophenyl, 2-, 3- or 4-methylphenyl, 2,3- or 3,5-dimethylphenyl, 2-, 3- or 4-methoxyphenyl and 4-methoxy-3,5-dimethylphenyl. Substituted or unsubstituted heteroaryl groups such as pyridyl may also be used. In one embodiment, each R^{19b} is independently a methyl, phenyl or substituted phenyl group.

In preferred processes of the invention, the catalyst is selected from:

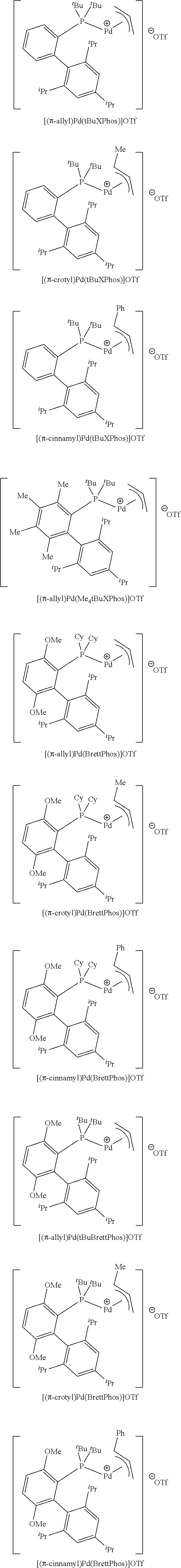

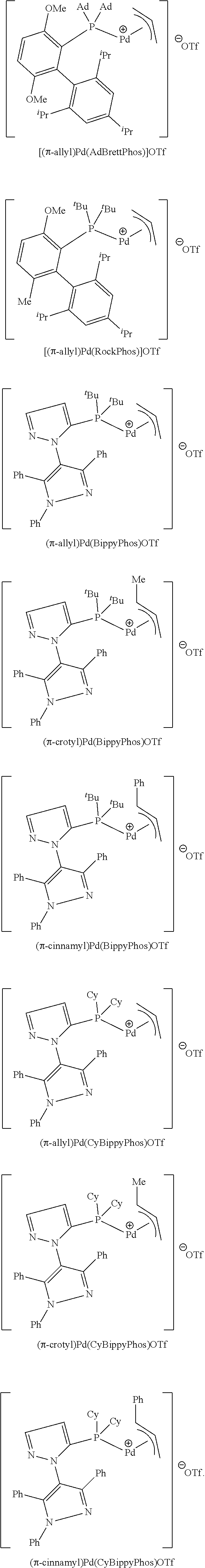

In preferred processes of the invention, the catalyst is a catalyst of formula (A), (B), (Ca), or (Cb), preferably a catalyst of formula (A).

In particularly preferred processes of the invention, the catalyst is [Pd(RuPhos)(crotyl)Cl] or [Pd(dippf)Cl₂]. More preferably, the catalyst is [Pd(dippf)Cl₂].

It has surprisingly been found that [Pd(dippf)Cl₂] and palladium complexes comprising Buchwald type ligands, e.g. [Pd(RuPhos)(crotyl)Cl], are able to catalyse the reaction between compounds of Formula I and compounds of Formula II under mild conditions, producing compounds of Formula III in high yields.

It may be preferred that the catalyst does not comprise crotyl or allyl ligands. It has been surprisingly found that palladium catalysts which do not comprise allyl or crotyl ligands, such as [Pd(dippf)Cl₂], are especially active in catalysing the reaction between compounds of Formula I and compounds of Formula II and such catalysts produce compounds of Formula III in superior yields. Advantageously, this means that lower loadings of catalyst may be used. Without being bound by theory it is believed that this is because [Pd(dippf)Cl₂] has an exemplary balance of steric and electronic properties particularly suited to performing the coupling reaction of the process of the invention.

The catalyst may be present in an amount of 0.1 mol % or more, 0.2 mol % or more, 0.3 mol % or more, or 0.4 mol % or more based upon the total amount of the compound of Formula I. The catalyst may be present in an amount of 3 mol % or less, 2.8 mol % or less, 2.6 mol % or less, or 2.5 mol % or less based upon the total amount of the compound of Formula I. The catalyst may be present in an amount of 0.1 mol % to 3 mol %, 0.2 to 2.8 mol %, 0.3 to 2.6 mol %, or 0.4 to 2.5 mol % based upon the total amount of the compound of Formula I. Typically, the catalyst is present in an amount of about 0.3-0.5 mol %, for example about 0.5 mol %, based upon the total amount of the compound of Formula I. However, higher catalyst loadings within these ranges may also be advantageous. Without wishing to be bound by theory, it is thought that the use of higher catalyst loadings within these ranges decreases the amount of impurities formed during the coupling reaction (e.g. as a result of deboronation of the compound of Formula II) and therefore allows for easier purification of the reaction mixture. Thus, in particularly preferred processes of the invention, the catalyst is present in an amount of at least 2.0 mol % based upon the total amount of the compound of Formula I, more preferably at least 2.25 mol % based upon the total amount of the compound of Formula I, even more preferably at least 2.5 mol % based upon the total amount of the compound of Formula I. For example, it may be preferred that the catalyst is present in an amount of 2.0 to 3.0 mol % based upon the total amount of the compound of Formula I.

The process of the invention comprises water. The amount of water present in the process of the invention may depend upon the choice of solvent. The skilled person will be able to select an appropriate amount of water to use in the process of the invention.

Typically, when an alcohol (e.g. iso-propyl and/or tert-amyl alcohol) is used as a solvent in the process of the invention water may be present in an amount of 5 molar equivalents or more, 6 molar equivalents or more, 7 molar equivalents or more, or 8 molar equivalents or more relative to the compound of Formula I. Typically, water may be present in an amount of 30 molar equivalents or less, 25 molar equivalents or less, 22 molar equivalents or less, or 20 molar equivalents or less. For example, water may be present in an amount of from 5 to 30 molar equivalents, from 6 to 25 molar equivalents, from 7 to 22 molar equivalents, or from 8 to 20 molar equivalents relative to the compound of Formula I.

Typically, when THF is used as a solvent in the process of the invention, a higher amount of water may be required. For example when THF is used as a solvent in the process of the invention water may be present in an amount of from 100 to 150 molar equivalents relative to the compound of Formula I.

As would be understood by a skilled person, water may be present in the process of the invention as incipient water in the solvent. That is to say, water may not need to be added and may already be comprised in the solvent, for example a so called “wet solvent”.

Without being bound by any sort of theory, it is believed that the presence of water may activate the —B(OR¹)(OR²) group bonded to Y in the compound of Formula II, and/or the carbonate group (i.e. the —OCO₂R group) of the compound of Formula I.

The process of the invention comprises a first base.

Preferably, the first base is selected from an alkali metal alkoxide, an alkali metal carbonate, an alkali metal phosphate, an alkali metal hydroxide, and an amine base. More preferably, the first base is selected from LiO^{t}Bu, NaO^{t}Bu, KO^{t}Bu, Na₂CO₃, K₂CO₃, Na₃PO₄, K₃PO₄, NaOH, KOH, Et₃N, Et₂ⁱPrN, DMAP, DABCO, or DBU. Most preferably, the first base is K₂CO₃ or KO^{t}Bu.

In some preferred processes of the invention, the first base is an alkoxide base (e.g. a metal alkoxide). For example, it may be preferred that the first base is a metal alkoxide, more preferably an alkali metal alkoxide. Even more preferably, the first base is selected from LiO^{t}Bu, NaO^{t}Bu, and KO^{t}Bu. Most preferably, the first base is NaO^{t}Bu.

In some preferred processes of the invention, the first base is selected from a metal carbonate, a metal phosphate, a metal hydroxide, and an amine base. Preferably, the first base is selected from an alkali metal carbonate, an alkali metal phosphate, an alkali metal hydroxide, and an amine base. More preferably, the first base is selected from Na₂CO₃, K₂CO₃, Na₃PO₄, K₃PO₄, NaOH, KOH, Et₃N, Et₂ⁱPrN, DMAP, DABCO, or DBU. Most preferably, the first base is K₂CO₃.

In preferred processes of the invention, the first base is a metal carbonate, preferably an alkali metal carbonate. In particularly preferred processes of the invention, the first base is selected from Na₂CO₃ and K₂CO₃. Most preferably, the first base is K₂CO₃.

In preferred processes of the invention, the first base is a metal phosphate, preferably an alkali metal phosphate. In particularly preferred processes of the invention, the first base is selected from Na₃PO₄ and K₃PO₄. Most preferably, the first base is K₃PO₄.

In preferred processes of the invention, the first base is a metal hydroxide, preferably an alkali metal hydroxide. In particularly preferred processes of the invention, the first base is selected from NaOH and KOH. Most preferably, the first base is KOH.

In preferred processes of the invention, the first base is an amine base. Preferably, the amine base is compound having a formula selected from R′—NH₂, R′₃NH, and R′₃N, wherein R′ are independently alkyl, aryl or heteroaryl groups, or the amine base is a cyclic amine base. In particularly preferred processes of the invention, the first base is selected from Et₃N, Et₂ⁱPrN, DMAP, DABCO, or DBU. Most preferably, the first base is Et₃N.

Typically, the first base is not a metal alkoxide in the process of the first aspect of the invention.

The first base is typically present in an amount of 200 mol % or more based on the total amount of the compound of Formula I, for example 250 mol % or more, 300 mol % or more, or 350 mol % or more based on the total amount of the compound of Formula I. The first base is typically present in an amount of 1000 mol % or less based on the total amount of the compound of Formula I, for example 800 mol % or less, 700 mol % or less, or 600 mol % or less based on the total amount of the compound of Formula I.

Optionally, and in preferred processes of the invention, the reacting of the compound of Formula I with the compound of Formula Il is carried out in the presence of a Lewis acid.

The Lewis acid may be a metal halide, preferably a lithium halide, such as lithium chloride, lithium bromide, or lithium iodide. Preferably the Lewis acid is lithium bromide or lithium iodide. Advantageously, lithium bromide and lithium iodide have increased solubility in organic solvents.

Typically, the Lewis acid is present in at least 1 molar equivalent relative to the compound of Formula I. For example, it may be preferred that the Lewis acid is present in at least 1.1, at least 1.2, at least 1.3, or at least 1.5 molar equivalents relative to the compound of Formula I. There is no particular upper limit for the amount of the Lewis acid which may be present. Typically, the Lewis acid is present in at most 4 molar equivalents relative to the compound of Formula I. For example it may be preferred that the Lewis acid is present in an at most 3.5, at most 3, at most 2.5, or at most 2, molar equivalents relative to the compound of Formula I. Preferably, the Lewis acid is present in the range of from 1 to 4 molar equivalents relative to the compound of Formula I, such as from 1.1 to 3.5 molar equivalents, 1.2 to 3 molar equivalents, 1.3 to 2.5 molar equivalents, or 1.5 to 2 molar equivalents, for example about 1 or about 1.5 molar equivalents, relative to the compound of Formula I.

The presence of a Lewis acid in the reaction between the compound of Formula I and the compound of Formula II has been found to increase the yield of the reaction compared to when the reaction is carried out under the same conditions in the absence of a Lewis acid. Furthermore, the presence of a Lewis acid in the reaction between the compound of Formula I and the compound of Formula II has been found to enable a larger number of compounds of Formula I to be used. In particular, the presence of a Lewis acid allows the use of certain compounds of Formula I, such as those where X is a heteroaryl comprising an sp² nitrogen atom and Z is bonded to a carbon atom of the heteroaryl adjacent to the sp² nitrogen atom of the heteroaryl, to be used in preparing a compound of Formula III. Without being bound by any sort of theory, it is believed that the presence of a Lewis acid may prevent the formation of a catalyst intermediate comprising a stable metal-allyl interaction which may deactivate the catalyst. Such an intermediate is shown in the illustrative example of Scheme 1.

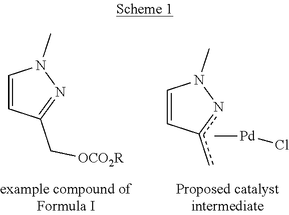

In preferred processes of the invention, the reacting of the compound of Formula I with the compound of Formula II is carried out in a solvent. The solvent may be an ether (e.g. tetrahydrofuran, methyltetrahydrofuran), an aromatic solvent (e.g. toluene), an alkyl solvent (e.g. heptane), an alcohol (e.g. iso-propanol or tert-amyl alcohol), a dialkylcarbonate (e.g. dimethylcarbonate), or a mixture thereof. Preferably, the solvent is an alcohol or mixture of alcohols, or a dialkylcarbonate. More preferably, the solvent is iso-propanol and/or tert-amyl alcohol, or dimethylcarbonte. Even more preferably, the solvent is iso-propanol and/or tert-amyl alcohol.

It may be preferred that the process of the invention is carried out in a solvent which is a dialkylcarbonate, such as dimethylcarbonate. When the solvent is a dialkylcarbonate (e.g. dimethylcarbonate) it has been surprisingly found that the yield of the compound of Formula III is increased. Without being bound by any sort of theory it is believed that this is because a dialkylcarbonate (e.g. dimethylcarbonate) may help reform the compound of Formula I if the compound of Formula I partially decomposes during the process (e.g. the compound of Formula I decomposes to form a corresponding alcohol, such as an alcohol of Formula IV as described hereinbelow).

It may be preferred that the process of the invention is carried out in a solvent which is an alcohol, such as iso-propyl and/or tert-amyl alcohol. Alcohol solvents, such as iso-propyl and/or tert-amyl alcohol, allow the amount of water in the reaction to be lowered, and have been surprisingly been found to provide higher yields of the compound of Formula III when X in the compound of Formula I is a heteroaryl group.

In preferred processes of the invention, the reacting of the compound of Formula I and the compound of Formula II is conducted at a temperature of greater than 60° C., greater than 65° C., greater than 70° C., or greater than 75° C. In preferred processes of the invention, the reacting of the compound of Formula I and the compound of Formula II is conducted at a temperature of less than 130° C., less than 120° C., less than 110° C., less than 100° C., or less than 85° C. For example, In preferred processes of the invention, the reacting of the compound of Formula I and the compound of Formula II is conducted at a temperature in the range of 60 to 130° C., 65 to 120° C., 70 to 110° C., 75 to 100° C., or 75 to 85° C.

It has surprisingly been found that an improved yield of the compound of Formula III may be obtained when the process of the invention is carried out at a temperature of 75 to 85° C. Surprisingly, a lower yield of the compound of Formula III may be obtained if the temperature is increased above 85° C. or below 75° C.

In preferred processes of the invention, the reacting of the compound of Formula I and the compound of Formula II is conducted for a duration of 1 hour or more, 2 hours or more, 3 hours or more, or 4 hours or more. In preferred processes of the invention, the reacting of the compound of Formula I and the compound of Formula II is conducted for a duration of 20 hours or less, 19 hours or less, 18 hours or less, or 17 hours or less. In preferred processes of the invention, the reacting of the compound of Formula I and the compound of Formula II is conducted for a duration of from 1 to 20 hours, 2 to 19 hours, 3 to 18 hours, or 4 to 17 hours. The skilled person will be able to select a suitable reaction duration and knows of techniques for monitoring the progress of the reaction (e.g. using high performance liquid chromatography).

It may be desirable to carry out the process of the invention under a blanket of an inert gas. Typically, the inert gas is nitrogen or argon.

In the process of the invention the sp²-hybridised carbon of Y in the compound of Formula II, to which the —B(OR¹)(OR²) group is bound, is the sp²-hybridised carbon atom at which the sp²-sp³ bond is ultimately formed. During the coupling reaction, the carbonate group (—OCO₂R) of the compound of Formula I functions as a leaving group and is eliminated during the reaction of the compound of Formula I and the compound of Formula II. The sp²-sp³ carbon-carbon bond is formed between the sp²-hybridised carbon atom of the compound of Formula II to which the boron atom was bonded and the sp³-hybridised carbon atom C* of Z, of the compound of Formula I.

The compounds of Formula II may be prepared in a borylation reaction involving reacting a borylation agent with a precursor to the compound of Formula II. The precursor to the compound of Formula II is the corresponding non-borylated compound (e.g. a compound of formula Y-H, wherein Y is as hereinbefore defined). The borylation agent used to functionalise the precursor to the compound of Formula II is not particularly limited and may be selected by the person skilled in the art. Preferably, the borylation agent is selected from B₂pin₂, HBpin, (dihydroxyboranyl)boronic acid, HBcat, and bis(catecholato)diboron. More preferably, the borylation agent is selected from B₂pin₂ and HBpin. Most preferably, the borylation agent is B₂pin₂. The skilled person is aware of suitable borylation reactions for preparing compounds of Formula II. For example, compounds of Formula II may be prepared according to a process known in the art, such as the one described in J. Org. Chem. 2009, 74, 23, 9199-9201, which is incorporated herein by reference in its entirety.

### Process for Preparing a Compound of Formula I

The process of the invention comprises the step of providing the compound of Formula I. In a preferred process of the invention, according to the second aspect of the invention, the process comprises the step of providing the compound of Formula I by reacting a compound of Formula IV,

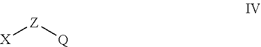

wherein X and Z are as hereinbefore defined; and
Q is hydroxy, or —OM where M is a metal;
with a dialkylcarbonate of formula RO(CO)OR, wherein each R is a substituted or unsubstituted C₁₋₂₀ straight-chain or C₃₋₂₀ branched-chain alkyl group, in the presence of a second base.

In general, using a compound of Formula IV to provide the compound of Formula I has the advantage of using a non-toxic, environmentally friendly reagent (i.e. a dialkylcarbonate) which reacts with a compound of Formula IV to give a compound of Formula I. Furthermore, a compound of Formula IV may be more easily sourced than a compound of Formula I, for instance a compound of Formula IV may be more easily sourced from a commercial supplier.

In the compound of Formula IV, Q is hydroxy, or —OM where M is a metal. More preferably Q is hydroxy, or —OM where M is an alkali or alkali earth metal. Most preferably, Q is hydroxy. Where Q is —OM where M is a metal, the metal may be selected from the list comprising Li, Na, K, Mg, Ca, and Ba. Where Q is —OM where M is a metal, the metal may be selected from the list comprising Li, Na, and K, more preferably Na and K.

In the dialkylcarbonate of formula RO(CO)OR, R is as generally described above. In preferred processes of the invention, the dialkylcarbonate of formula RO(CO)OR is dimethylcarbonate, diethylcarbonate, di-iso-propycarbonate, or di-tert-butylcarbonate. Preferably, the dialkylcarbonate is dimethylcarbonate.

Typically, the dialkylcarbonate is used as both a solvent and a reagent. Typically, the dialkyl carbonate is present in excess relative to the compound of Formula IV. The amount of dialkylcarbonate in the process of the invention is not particularly limited. Typically, the dialkylcarbonate is present in an amount of greater than or equal to 15 equivalents, greater than or equal to 20 equivalents, greater than or equal to 25, or greater than or equal to 30 equivalents based upon the total amount of the compound of Formula IV. Typically, the dialkylcarbonate is present in an amount of less than or equal to 100 equivalents, less than or equal to 80 equivalents, less than or equal to 7015 equivalents , or less than or equal to 60 equivalents based upon the total amount of the compound of Formula IV. For example, typically the invention, the dialkylcarbonate is present in an amount of from 15 to 100 equivalents, from 20 to 80 equivalents, from 25 to 70 equivalents %, or from 30 to 60 equivalents based upon the total amount of the compound of Formula IV.

In preferred processes of the invention, the second base is a metal carbonate, a metal alkoxide, or a metal phosphate. Preferably, the second base is potassium carbonate, sodium-tert-butoxide, or potassium phosphate, most preferably potassium carbonate.

In preferred processes of the invention, the second base is a metal alkoxide. The metal alkoxide is preferably a metal methoxide, a metal ethoxide, a metal iso-propoxide, or a metal tert-butoxide.

In preferred processes of the invention, the second base is an alkali metal alkoxide. The alkali metal alkoxide is preferably an alkali metal methoxide, an alkali metal ethoxide, an alkali metal iso-propoxide, or an alkali metal tert-butoxide. The alkali metal alkoxide is more preferably an alkali metal methoxide, an alkali metal ethoxide, or an alkali metal tert-butoxide, preferably an alkali metal tert-butoxide. Preferred metal alkoxides include sodium methoxide, sodium ethoxide, sodium tert-butoxide, potassium methoxide, potassium ethoxide, or potassium tert-butoxide, preferably sodium tert-butoxide or potassium tert-butoxide.

The metal alkoxide may be formed in-situ by reaction of a corresponding alcohol with a third base, for example a metal tert-butoxide may be formed in-situ by reaction between tert-butanol and a metal hydroxide, a metal carbonate, a metal phosphate, or a metal hydrogenphosphate.

In preferred processes of the second aspect of the invention, the second base is present in an amount of 200 mol % or more relative to the compound of Formula IV, 220 mol % or more relative to the compound of Formula IV, or 240 mol % or more relative to the compound of Formula IV. The second base may be present in an amount of 300 mol % or less relative to the compound of Formula IV, 280 mol % or less relative to the compound of Formula IV, or 260 mol % or less relative to the compound of Formula IV. For Example, the second base may be present in an amount of from 200 to 300 mol % relative to the compound of Formula IV, 220 to 280 mol % relative to the compound of Formula IV, or 240 to 260 mol % relative to the compound of Formula IV.

In preferred processes for providing a compound of Formula I, the compound of Formula IV is reacted at a temperature in the range 70 to 85° C., preferably 70 to 80° C., more preferably 75 to 80° C.

In preferred processes for providing a compound of Formula I, the compound of Formula IV is reacted with the dialkylcarbonate to give a compound of Formula I for a duration of 1 to 16 hours, preferably 2 to 14 hours, more preferably 3 to 12 hours, most preferably 4 to 10 hours.

### In-Situ Process

In a preferred process of the invention, the process comprises the step of providing the compound of Formula I in-situ, according to the third aspect of the invention. Providing the compound of Formula I in-situ gives the so called in-situ process of the invention. The in-situ process of the invention comprises using a compound of Formula IV and a compound of Formula II as starting materials, and removes the need to provide a compound of Formula I directly as a starting material. In the in-situ process of the invention the compound of Formula I is formed in-situ from the compound of Formula IV. As will be understood, the compound of Formula I which is produced in-situ reacts with the compound of Formula II to produce the compound of Formula III in the same fashion as described hereinabove.

Accordingly, in the in-situ process of the invention the compound of Formula I is provided in-situ in the presence of the compound of Formula II, the catalyst, the water, and the first base by reacting a compound of Formula IV

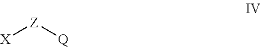

wherein X and Z are as hereinbefore defined; and
Q is hydroxy, or —OM where M is a metal;
with a dialkylcarbonate of formula RO(CO)OR, wherein each R is a substituted or unsubstituted C₁₋₂₀ straight-chain or C₃₋₂₀ branched-chain alkyl group.

Advantageously, the in-situ process of the invention means a compound of Formula I does not have to be supplied, as such, to the reaction of the invention. Instead, the compound of Formula I is formed in-situ from a compound of Formula IV. A compound of Formula IV may be more easily sourced than a compound of Formula I, for instance a compound of Formula IV may be more easily sourced from a commercial supplier. In general, using a compound of Formula IV to prepare the compound of Formula I has the advantage of using a non-toxic, environmentally friendly reagent, a dialkylcarbonate, which reacts with a compound of Formula IV to give a compound of Formula I.

The invention further provides a process for forming an sp²-sp³ carbon-carbon bond, the process comprising:
providing a compound of Formula I

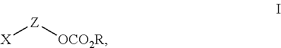

wherein X is a substituted or unsubstituted aromatic group;
Z is an organic group comprising an sp³-hybridised carbon atom C* and having formula —C*(H)(R³)— where R³ is H, OH, a substituted or unsubstituted alkyl group, a substituted or unsubstituted alkenyl group, or a substituted or unsubstituted aromatic group;
R is a substituted or unsubstituted C₁₋₂₀ straight-chain or C₃₋₂₀ branched-chain alkyl group; and
reacting the compound of Formula I with a compound of Formula II

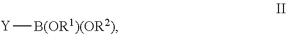

wherein Y is a substituted or unsubstituted aromatic group; and
R¹ and R² are, independently, H or a substituted or unsubstituted organic group comprising 1-20 carbon atoms; or, together with the atoms to which they are attached, R¹ and R² form a ring;
in the presence of a catalyst, water, and a first base, and optionally a Lewis acid, to give a compound of Formula III:

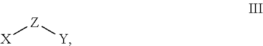

wherein X, Z and Y are as hereinbefore defined; and
wherein:
in the compound of Formula II, the boron atom is bonded to Y at an sp²-hybridised carbon atom in Y; and
in the compound of Formula III, the sp³-hybridised carbon atom C* of Z, is bonded to Y at said sp²-hybridised carbon atom in Y, and
the compound of Formula I is provided in-situ in the presence of the compound of Formula II, the catalyst, the water, and the first base by reacting a compound of Formula IV

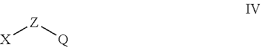

wherein X and Z are as hereinbefore defined; and
Q is hydroxy, or —OM where M is a metal;
with a dialkylcarbonate of formula RO(CO)OR, wherein each R is a substituted or unsubstituted C₁₋₂₀ straight-chain or C₃₋₂₀ branched-chain alkyl group.

It is a further advantage of the in-situ process of the invention that it has been found to proceed with the formation of zero, or minimal, by-products. It has surprisingly been found that by preparing the compound of Formula I in-situ from a compound of Formula IV, compounds of Formula I and compounds of Formula III are substantially the only compounds formed (i.e. minimal or no unwanted by-products are formed). Furthermore, the in-situ process of the invention has the advantage that the dialkylcarbonate of formula RO(CO)OR may serve the role of both reagent and solvent.

Accordingly, the in-situ process may be carried out in the absence of a solvent other than the dialkylcarbonate. For the avoidance of doubt, and to the extent that the dialkylcarbonate of formula RO(CO)OR may be considered a solvent, the in-situ process may be carried out in the absence of a solvent other than the dialkylcarbonate of formula RO(CO)OR.

In the compound of Formula IV, Q is hydroxy, or —OM where M is a metal. More preferably Q is hydroxy, or —OM where M is an alkali or alkali earth metal. Most preferably, Q is hydroxy. Where Q is —OM where M is a metal, the metal may be selected from the list comprising Li, Na, K, Mg, Ca, and Ba. Where Q is —OM where M is a metal, the metal may be selected from the list comprising Li, Na, and K.

In the dialkylcarbonate of formula RO(CO)OR, R is as generally described above. In preferred in-situ processes of the invention, the dialkylcarbonate of formula RO(CO)OR is dimethylcarbonate, diethylcarbonate, di-iso-propylcarbonate, or di-tert-butylcarbonate. Preferably, the dialkylcarbonate is dimethylcarbonate.

In preferred in-situ processes of the invention, the dialkylcarbonate is present in an amount of greater than or equal to 1000 mol %, greater than or equal to 1300 mol %, greater than or equal to 1500 mol %, or greater than or equal to 1700 mol % based upon the total amount of the compound of Formula IV. In preferred processes of the invention, the dialkylcarbonate is present in an amount of less than or equal to 3600 mol %, less than or equal to 3400 mol %, less than or equal to 3200 mol %, or less than or equal to 3000 mol % based upon the total amount of the compound of Formula IV. For example, in preferred processes of the invention, the dialkylcarbonate is present in an amount of from 1000 to 3600 mol %, from 1300 to 3400 mol %, from 1500 to 3200 mol %, or from 1700 to 3000 mol % based upon the total amount of the compound of Formula IV.

The in-situ process of the invention comprises a first base. The first base may be as described above. For example, the first base may be a metal carbonate or a metal phosphate. Preferably, the first base is potassium carbonate or potassium phosphate, most preferably potassium carbonate.

Additionally, in the in-situ process of the invention, the first base may be a metal alkoxide. It may be preferred that in the in-situ process of the invention the first base is a metal alkoxide. Preferably, the first base may be NaO^{t}Bu or KO^{t}Bu.

It has surprisingly been found that in the in-situ process of the invention that a metal alkoxide may be preferably used as the first base. It has surprisingly been found in the in-situ process of the invention that using a metal alkoxide as the first base may give a superior yield of the compound of Formula III as compared to when a metal carbonate or metal phosphate is used as a base.

In preferred in-situ processes of the invention, the process is carried out at a temperature in the range 50 to 100° C., preferably 60 to 95° C., more preferably 65 to 90° C., more preferably 70 to 85° C.

In preferred in-situ processes of the invention, the process is carried out for a duration of 1 to 16 hours, preferably 2 to 14 hours, more preferably 3 to 12 hours, most preferably 4 to 10 hours.

### EXAMPLES

### General Procedure for Preparing Compounds of Formula I (General Procedure I)

To a flask, a compound of Formula IV (25 mmol, 1 equivalent) and dimethyl carbonate (53.5 g, 50 mL, 0.5 M) was charged. A Dean Stark trap and a condenser was attached to the flask to form a reactor. The reactor was sealed with a septa and nitrogen was allowed to flow through. The reactor was warmed to 90° C. and a solution of sodium methoxide in methanol was charged into the flask by syringe (0.29 mL, 4.3 M, 0.05 equivalence). The reactor was warmed to reflux. The distillate was collected in the trap for 30 minutes. Once the Dean Stark trap was filled with distillate it was discarded and the trap was filled with dimethyl carbonate (15 mL). The solvent was allowed to reflux for 30 minutes, and then the solvent in the trap was removed. This process was repeated for 3 hours before full conversion was reached as identified by ¹H NMR. The reactor was cooled, the organic layer was separated, washed with brine 3 times, and dried over Na₂SO₄. The dried organic layer was filtered through celite and the solvent evaporated to yield a compound of Formula I.

### General Procedure for Reactions of Compounds of Formula I with Compounds of Formula II to Produce a Compound of Formula III (General Procedure II)

To a flask was charged a palladium catalyst [Pd(RuPhos)(crotyl)Cl] or [Pd(dippf)Cl₂] (0.1-1 mol % based upon the compound of Formula I or IV), a compound of Formula I or a compound of Formula IV (in the case of the in-situ process) (1 eq), a base (K₂CO₃ or NaO^{t}Bu, 2 eq), a compound of Formula II (1.1 eq), and optionally a Lewis acid LiBr (1 eq). The flask was sealed with a septum. The atmosphere was evacuated and refilled with nitrogen three times. Solvent, and optionally water, was added to the flask. With constant stirring, the contents of the flask were heated to the desired temperature for the required period of time before being cooled, and then extracted with ethyl acetate. The ethyl acetate extracts were combined and evaporated to yield a crude solid. The resulting crude solid was analysed by ¹H NMR which was used to calculate the conversion to the compound of Formula III. Purification of the compound of Formula III was achieved by recrystallisation in 0-10% methanol/dichloromethane or 0-50% methyl-tert-butyl ether in heptanes allowing a yield for the process to be obtained. Alternatively, column chromatography was used to purify the compound of Formula III.

### Materials

[Pd(RuPhos)(crotyl)Cl] and [Pd(dippf)Cl₂] are commercially available from Johnson Matthey. Compounds of Formula I were prepared according to General Procedure I, above, from the corresponding compound of Formula IV. Compounds of Formula II and compounds of Formula IV were obtained commercially from Combi Blocks and TCI America.

Methyl carbonate, K₂CO₃, LiBr, and all solvents are available commercially from Combi Blocks and TCI America. Solvents were degassed by sparging with nitrogen gas for at least 2 hours before use.

### Measurement Methods

Nuclear magnetic resonance (NMR) measurements were conducted using a Bruker 400 MHz NMR spectrometer.

### Experiment 1

For each of Examples 1-37 listed in Table 1, reaction conditions a-h were employed and are denoted by the superscript letter by the example number (e.g. 1^{a} means that Example 1 was conducted under reaction conditions a). The reaction conditions a-h are summarised in Table 2. For each of Examples 1-37 listed in Table 1, the boron group B(OR)₂ in the compounds of Formula II was B(OH)₂. For each of Examples 1-37 the catalyst used was [Pd(RuPhos)(crotyl)Cl].

Compounds of Formula I were prepared according to General procedure I, above, from the corresponding alcohols of Formula IV. Compounds of Formula I were reacted with compounds of Formula II to form compounds of Formula III according to General Procedure II described above. Table 1 shows the compounds of Formula I, II, and III, and the yield of the reaction in each case. The sp²-sp³ carbon-carbon bond formed during the reaction is shown in bold in the structure of the compounds of Formula III given in Table 1.

TABLE 1

| | compound of | compound of | compound of Formula | Yield/ |
|---|---|---|---|---|
| Ex | Formula I | Formula II | III | % |
| 1ª | 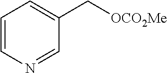 | 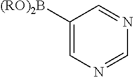 | 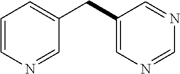 | 68 |
| 2ª | 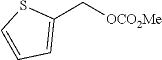 | 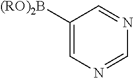 | 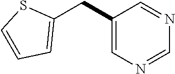 | 72 |
| 3ª | 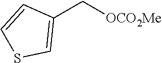 | 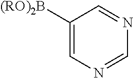 | 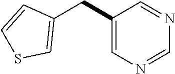 | 78 |
| 4ª | 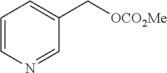 | 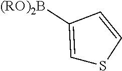 | 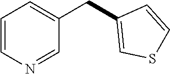 | 91 |
| 5ª | 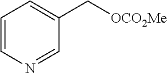 | 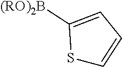 | 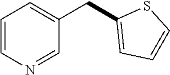 | 68 |
| 6ª | 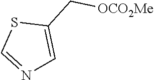 | 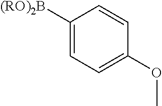 | 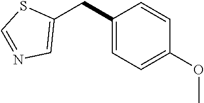 | 62 |
| 7ª | 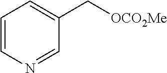 | 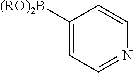 | 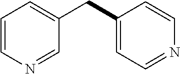 | 40 |
| 8ª | 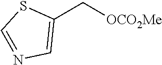 | 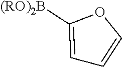 | 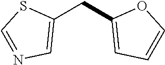 | 60 |
| 9ª | 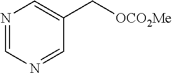 | 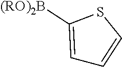 | 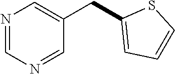 | 80 |
| 10^{a} | 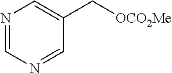 | 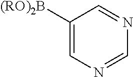 | 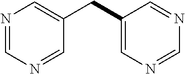 | 50 |
| 11ª | 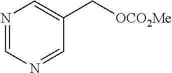 | 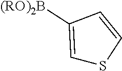 | 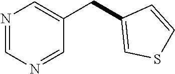 | 93 |
| 12^{b} | 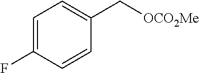 | 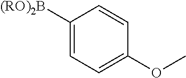 | 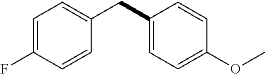 | 91 |
| 13^{b} | 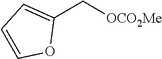 | 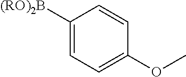 | 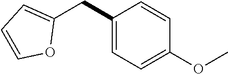 | 75 |
| 14^{b} | 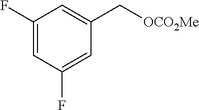 | 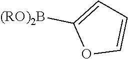 | 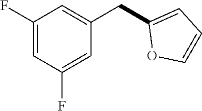 | 92 |
| 15^{b} | 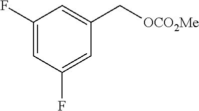 | 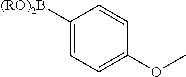 | 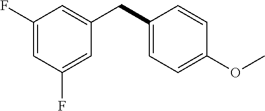 | 87 |
| 16^{b} | 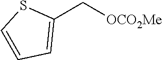 | 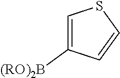 | 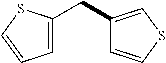 | 82 |
| 17^{b} | 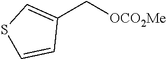 | 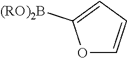 | 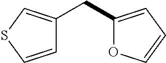 | 65 |
| 18^{b} | 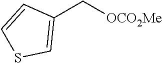 | 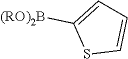 | 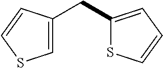 | 75 |
| 19^{b} | 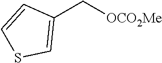 | 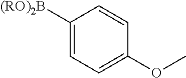 | 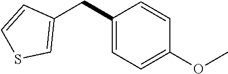 | 82 |
| 20^{b} | 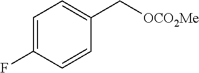 | 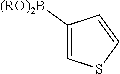 | 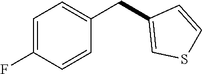 | 93 |
| 21^{c} | 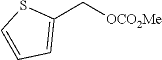 | 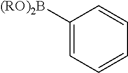 | 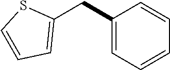 | 91 |
| 22^{b} | 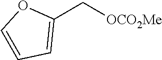 | 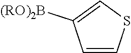 | 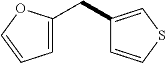 | 63 |
| 23^{b} | 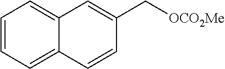 | 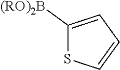 | 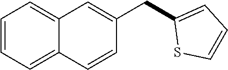 | 81 |
| 24^{d} | 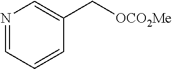 | 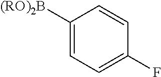 | 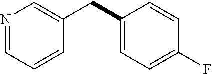 | 88 |
| 25^{d} | 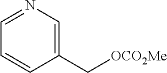 | 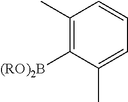 | 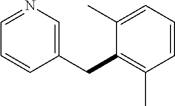 | 87 |
| 26^{c} | 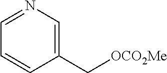 | 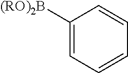 | 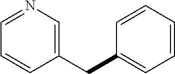 | 86 |
| 27^{d} | 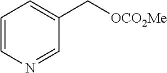 | 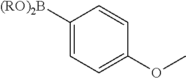 | 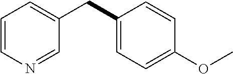 | 95 |
| 28^{d} | 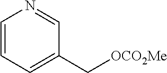 | 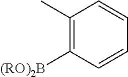 | 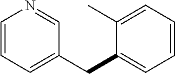 | 91 |
| 29^{d} | 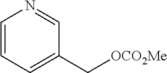 | 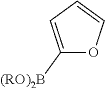 | 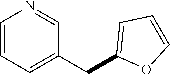 | 88 |
| 30^{d} | 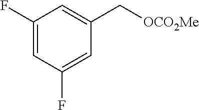 | 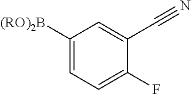 | 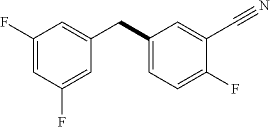 | 92 |
| 31^{d} | 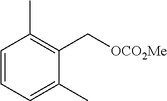 | 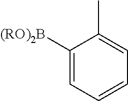 | 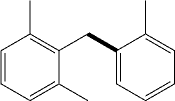 | 93 |
| 32^{e} | 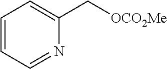 | 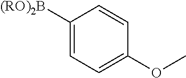 | 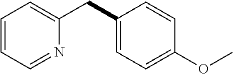 | 0 |
| 33^{f} | 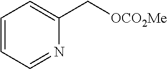 | 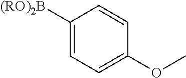 | 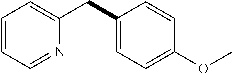 | 40 |
| 34^{g} | 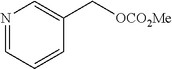 | 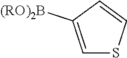 | 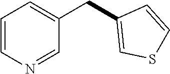 | 95 |
| 35^{g} | 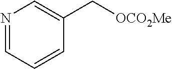 | 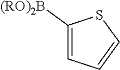 | 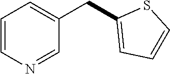 | 68 |
| 36^{h} | 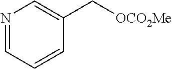 | 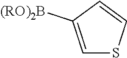 | 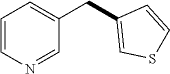 | 95 |
| 37^{h} | 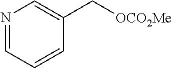 | 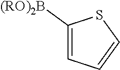 | 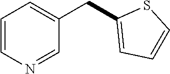 | 65 |

TABLE 2

| | Catalyst | | | | Temper- | | Lewis |
|---|---|---|---|---|---|---|---|
| Con- | loading*/ | K₂CO₃/ | H₂O/ | | ature/ | Time/ | Acid |
| dition | mol % | eq | eq | Solvent | ° C. | hours | (1 eq) |
| a | 0.5 | 2 | 20 | tert-amyl | 75 | 15 | — |
| | | | | alcohol | | | |
| b | 0.1 | 2 | 130- | THF | 65 | 15 | — |
| | | | 140 | | | | |
| c | 0.5 | 2 | 130- | THF | 65 | 15 | — |
| | | | 140 | | | | |
| d | 0.3 | 2 | 130- | THF | 65 | 15 | — |
| | | | 140 | | | | |
| e | 1.0 | 2 | 20 | iso- | 100 | 15 | — |
| | | | | propanol | | | |
| f | 0.1 | 2 | 20 | iso- | 100 | 15 | LiBr |
| | | | | propanol | | | |
| g | 0.5 | 2 | 20 | tert-amyl | 100 | 15 | — |
| | | | | alcohol | | | |
| h | 0.3 | 2 | 20 | tert-amyl | 100 | 15 | LiBr |
| | | | | alcohol | | | |

*Catalyst loading is relative to the total amount of the compound of Formula I.

Examples 1-37 show that the process of the invention can be applied to a broad range of starting material substrates. In particular, the process of the invention can be successfully used to couple heteroaryl and/or aryl compounds and is effective even where the starting materials are sterically hindered.

The Examples further show that the process of the invention can be conducted under mild conditions. For example, catalyst loadings as low as 0.1 mol % and temperatures as low as 65° C. give excellent yields.

Examples 32-37 demonstrate the effect of adding a Lewis acid to the reaction mixture. A comparison of Examples 32 and 33 shows that the addition of LiBr to the reaction mixture can increase the yield of the reaction, even at a lower catalyst loading (no reaction in Example 32 compared to a yield of 40% in Example 33). Furthermore, addition of LiBr in Examples 36 and 37 produced similar yields compared to Examples 34 and 35, respectively, despite the use of a lower catalyst loading.

### Experiment 2—In-Situ Process

Examples 38-41 were carried out according to General Procedure II described above, using a compound of Formula IV to generate a compound of Formula I in-situ. Dimethyl carbonate was present as both a reagent and solvent. Examples 38-41 demonstrate an in-situ synthesis of compounds of Formula III. Dimethyl carbonate (2 mmol) was added to the flask at the same time as the compound of Formula IV and the compound of Formula II. For each of Examples 38-41 the catalyst was [Pd(RuPhos)(crotyl)Cl].

For each of Examples 38-41 listed in Table 3, reaction conditions i, j, or k were employed and are denoted by the superscript letter by the example number (e.g. 38ⁱ means that Example 38 was conducted under reaction conditions i). The reaction conditions i, j and k are summarised in Table 4.

Table 3 shows the compounds of Formula IV, II, and III, and the yield of the reaction in each case. The sp²-sp³ carbon-carbon bond formed during the reaction is shown in bold in the structure of the compounds of Formula III given in Table 3.

TABLE 3

| | compound of | compound of | | Yield/ |
|---|---|---|---|---|
| Ex | Formula IV | Formula II | compound of Formula III | % |
| 38ⁱ | 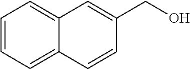 [SMILES: OCc1ccc2ccccc2c1] | 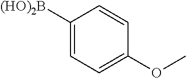 | 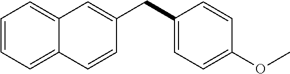 | 30 |
| 39ⁱ | 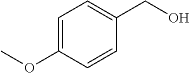 | 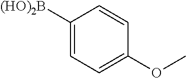 | 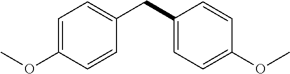 | 24 |
| 40ⁱ | 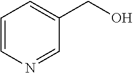 | 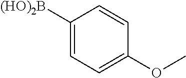 | 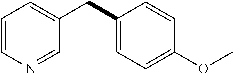 | 20 |
| 41^{j} | 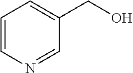 | 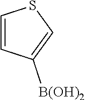 | 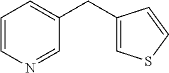 [SMILES: c1cncc(Cc2ccsc2)c1] | 50 |
| 42^{k} | 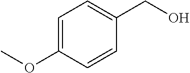 | 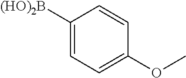 | 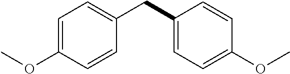 | 98 |

TABLE 4

| | Catalyst | | | | | Methyl |
|---|---|---|---|---|---|---|
| Con- | loading*/ | | | Temp/ | Time/ | carbonate/ |
| dition | mol % | Base/eq | H₂O/eq | ° C. | hours | eq |
| i | 0.5 | 2(K₂O₃) | 20 | 75 | 15 | 59 |
| j | 0.5 | 2(K₂O₃) | 20 | 100 | 15 | 59 |
| k | 1 | 2(NaO^{t}Bu) | incipient | 85 | 4-16 | 59 |

*Catalyst loading is relative to the total amount of the compound of Formula I generated from the compound of Formula IV, assuming 100% conversion.

The crude reaction mixture of Example 41 was analysed by ¹H NMR spectrometry to calculate the conversion. The % amounts of each compound present in the crude reaction mixture is shown in Table 5.

TABLE 5

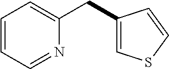

50%

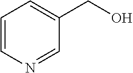

<1%

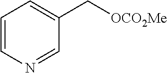

50%

Examples 38-42 show the broad applicability of the in-situ process of the invention to a number of different substrates. More specifically, Examples 38-42 show how compounds of Formula IV can be directly transformed into compounds of Formula III in-situ under the coupling reaction conditions when a dialkylcarbonate is also present, and in excellent yields. This in-situ preparation of compounds of Formula I makes the process of the invention highly accessible and capable of coupling an array of commercially available or easily obtainable substrates without the need to form an alkyl carbonate containing compound of Formula I in a separate reaction step. Furthermore, the in-situ process shows high selectivity for the desired compound of Formula III in good to excellent yield with minimal, or zero, by-product formation. It is a further advantage of the in-situ process that no other compounds other than compounds of Formula I, compounds of Formula III, and compounds of Formula IV are present following the coupling reaction.

Example 42 uses NaO^{t}Bu as a base. Using NaO^{t}Bu as a base gives a vastly improved yield in the in-situ process of the invention as compared to when K₂CO₃ is used as a base (c.f. Example 39).

### Experiment 3—Alternative Carbonates and Catalysts

Examples 43 and 44, as shown in Table 6, were carried out according to the General Procedure II described above. In Example 43 and 44, the compound of Formula I was prepared using General Procedure I except that di-tert-butylcarbonate was used instead of dimethyl carbonate.

Examples 43 and 44 demonstrate the use of alternative carbonates in the compound of Formula I and the effect of the choice of catalyst. For each of Examples 43 and 44 listed in Table 6, reaction conditions I or m were employed and are denoted by the superscript letter by the example number (e.g. 431 means that Example 43 was conducted under reaction conditions I). The reaction conditions I and m are summarised in Table 7.

Table 6 shows the compounds of Formula I, II, and III, and the yield of the reaction in each case. The sp²-sp³ carbon-carbon bond formed during the reaction is shown in bold in the structure of the compounds of Formula III given in Table 6.

TABLE 6

| | compound of | | | |
|---|---|---|---|---|
| | Formula I or Formula | compound of | compound of | |
| Ex | IV | Formula II | Formula III | Yield/% |
| 43^{l} | 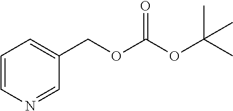 | 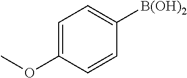 | 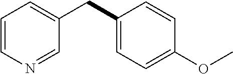 | 20% |
| 44^{m} | 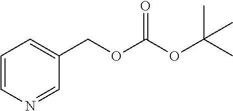 | 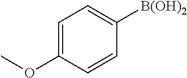 | 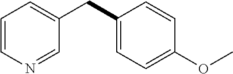 | 95% |

TABLE 7

| | Catalyst | | | | | |
|---|---|---|---|---|---|---|
| Con- | loading*/ | | | H₂O/ | Temp/ | Time/ |
| dition | mol % | Catalyst | Base/eq | eq | ° C. | hours |
| l | 1 | [Pd(RuPhos)(crotyl)Cl] | 2 eq of | 20 | 85 | 5 |
| | | | K₂CO₃ | | | |
| m | 1 | [Pd(dippf)Cl₂] | 2 eq of | 20 | 85 | 5 |
| | | | K₂CO₃ | | | |

*Catalyst loading is relative to the total amount of the compound of Formula I generated from the compound of Formula IV, assuming 100% conversion.

Examples 43 and 44 show that alternative carbonate groups may be used in the compound of Formula I and that improved yields may be achieved with a [Pd(dippf)Cl₂] catalyst.

### Experiment 4—Alternative Carbonates in the In-Situ Process

Examples 45, 46 and 47, as shown in Table 8, were carried out according to General Procedure II, described above, and a compound of Formula IV was used instead of a compound of Formula I. Either diethylcarbonate (Examples 45 and 46) or di-iso-propyl carbonate (Experiment 47) was present as both a reagent and solvent. As in Experiment 2, each dialkylcarbonate (2 mmol) was added to the flask at the same time as the compound of Formula IV and the compound of Formula II. For each of Examples 45, 46 and 47, the catalyst was [Pd(RuPhos)(crotyl)Cl].

For each of Examples 45, 46 and 47 listed in Table 8, reaction conditions n, o or p were employed and are denoted by the superscript letter by the example number (e.g. 46° means that Example 46 was conducted under reaction conditions o). The reaction conditions n, o and p are summarised in Table 9.

Table 8 shows the compounds of Formula IV, II, and III, and the yield of the reaction in each case. The sp²-sp³ carbon-carbon bond formed during the reaction is shown in bold in the structure of the compounds of Formula III given in Table 8.

TABLE 8

| | compound of | compound of | | |
|---|---|---|---|---|
| Ex | Formula IV | Formula II | compound of Formula III | Yield/% |
| 45ⁿ | 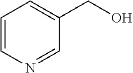 | 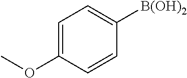 | 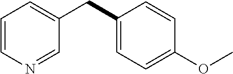 | >95% |
| 46^{o} | 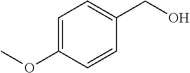 | 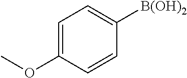 | 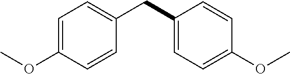 | >95% |
| 47^{p} | 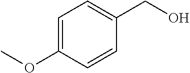 | 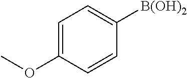 | 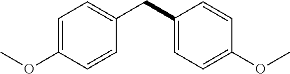 | >95% |

TABLE 9

| | Catalyst | | | | |
|---|---|---|---|---|---|
| | loading* / | | Temp / | Time / | |
| Condition | mol % | Base / eq | ° C. | hours | dialkylcarbonate |
| n | 1 | 2 eq of | 85 | 8 | ethyl |
| | | NaO^{t}Bu | | | |
| o | 1 | 2 eq of | 85 | 8 | ethyl |
| | | NaO^{t}Bu | | | |
| p | 1 | 2 eq of | 85 | 8 | iso-propyl |
| | | NaO^{t}Bu | | | |

*Catalyst loading is relative to the total amount of the compound of Formula I generated from the compound of Formula IV, assuming 100% conversion.

Experiment 4 shows that high yields of the compound of Formula III can be obtained using the in-situ process of the invention using a variety of dialkylcarbonates and when using different substrates (e.g. homoaryl and heteroaryl compounds).

1. A process for forming an sp²-sp³ carbon-carbon bond, the process comprising:
providing a compound of Formula I 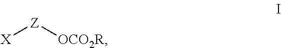
wherein X is a substituted or unsubstituted aromatic group;
Z is an organic group comprising an sp³-hybridised carbon atom C* and having formula —C*(H)(R³)— where R³ is H, OH, a substituted or unsubstituted alkyl group, a substituted or unsubstituted alkenyl group, or a substituted or unsubstituted aromatic group; and
R is a substituted or unsubstituted C₁₋₂₀ straight-chain or C₃₋₂₀ branched-chain alkyl group; and
reacting the compound of Formula I with a compound of Formula II 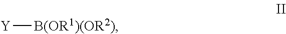
wherein Y is a substituted or unsubstituted aromatic group, and
R¹ and R² are, independently, H or a substituted or unsubstituted organic group comprising 1-20 carbon atoms; or, together with the atoms to which they are attached, R¹ and R² form a ring;
in the presence of a catalyst, water, a first base, and a Lewis acid, to give a compound of Formula III: 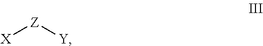
wherein X, Z and Y are as hereinbefore defined; and
wherein:
in the compound of Formula II, the boron atom is bonded to Y at an sp²-hybridised carbon atom in Y; and
in the compound of Formula III, the sp³-hybridised carbon atom C* of Z, is bonded to Y at said sp²-hybridised carbon atom in Y.

2. The process according to claim 1, wherein R³ is H, a substituted or unsubstituted aromatic group, or a substituted or unsubstituted C₁₋₂₀ alkyl group.

3. (canceled)

4. The process according to claim 1, wherein R in the compound of Formula I is a substituted or unsubstituted C₁₋₁₀ straight-chain or C₃₋₁₀ branched-chain alkyl group, preferably a substituted or unsubstituted C₁₋₄ straight-chain or C₃₋₄ branched-chain alkyl group.

5. The process according to claim 1, wherein X in the compound of Formula I is a substituted or unsubstituted aryl group or a substituted or unsubstituted heteroaryl group.

6-9. (canceled)

10. The process according to claim 1, wherein the compound of Formula I has a sub-Formula Ia 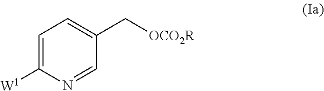
wherein R is a substituted or unsubstituted C₁₋₂₀ straight-chain or C₃₋₂₀ branched-chain alkyl group; and
W¹ is a substituent selected from -halo, —C(halo)₃, —Rₐ, —O—Rₐ, —S—Rₐ, —NRₐR_{b}, —CN, —NO₂, —C(O)—Rₐ, —COORₐ, —C(S)—Rₐ, —C(S)ORₐ, —S(O)₂OH, —S(O)₂—Rₐ,
—S(O)₂NRₐR_{b}, —O—S(O)—Rₐ and —CONRₐR_{b},
wherein Rₐ and R_{b} are independently selected from the groups consisting of H, substituted or unsubstituted alkyl, substituted or unsubstituted aryl, substituted or unsubstituted arylalkyl, substituted or unsubstituted heteroalkyl, substituted or unsubstituted heteroaryl, or Rₐ and R_{b} together with the atom to which they are attached form a heterocycloalkyl group.

11. The process according to claim 1, wherein R¹ and R² in the compound of Formula II are, independently, H or a substituted or unsubstituted C₁₋₂₀ straight-chain or C₃₋₂₀ branched-chain alkyl group, preferably H or a substituted or unsubstituted C₁₋₁₀ straight-chain or C₃₋₁₀ branched-chain alkyl group, more preferably H or a substituted or unsubstituted C₁₋₄ straight-chain or C₃₋₄ branched-chain alkyl group.

12. The process according to claim 1, wherein R¹ and R² in the compound of Formula II form a ring, preferably R¹ and R² form a ring which is —Bpin.

13. The process according to claim 1, wherein Y in the compound of Formula II is a substituted or unsubstituted aryl group, or a substituted or unsubstituted heteroaryl group

14. The process according to claim 1, wherein Y in the compound of Formula II is a substituted or unsubstituted C₆-C₂₀ aryl group, preferably a substituted or unsubstituted C₆-C₁₈ aryl group, more preferably a substituted or unsubstituted C₆-C₁₀ aryl group.

15. The process according to claim 14, wherein Y in the compound of Formula II is selected from substituted or unsubstituted phenyl, tolyl, xylyl, methoxyphenyl, difluorophenyl, anthracenyl, and naphthalenyl.

16. The process according to claim 1, wherein Y in the compound of Formula II is a substituted or unsubstituted C₄-C₂₀ heteroaryl group, preferably a substituted or unsubstituted C₄-C₁₈ heteroaryl group, more preferably a substituted or unsubstituted C₄-C₁₀ heteroaryl group.

17. The process according to claim 16, wherein Y in the compound of Formula II is selected from substituted or unsubstituted pyridinyl, pyrrolyl, imidazolyl, triazolyl, tetrazolyl, indolyl, pyridazinyl, pyrimidinyl, pyrazinyl, quinolinyl, benzimidazolyl, pyrazolyl, azaindolyl, furanyl, benzofuranyl, thiophenyl, benzothiophenyl, thiazolyl, isothiazolyl, thiadiazolyl, 1,2-benzthiazolyl, oxazolyl, isoxazolyl, oxadiazolyl, and benzoxazolyl.

18. The process according to claim 16, wherein the compound of Formula II has a sub-Formula IIa 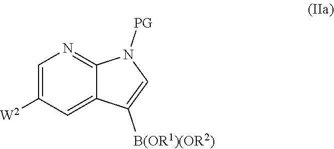
wherein R¹ and R² are, independently, H or a substituted or unsubstituted organic group comprising 1-20 carbon atoms; or, together with the atoms to which they are attached, R¹ and R² form a ring;
W² is a substituent selected from -halo, —C(halo)₃, —Rₐ, —O—Rₐ, —S—Rₐ, —NRₐR_{b}, —CN, —NO₂, —C(O)—Rₐ, —COORₐ, —C(S)—Rₐ, —C(S)ORₐ, —S(O)₂OH, —S(O)₂—Rₐ,
—S(O)₂NRₐR_{b}, —O—S(O)—Rₐ and —CONRₐR_{b},
wherein Rₐ and R_{b} are independently selected from the groups consisting of H, substituted or unsubstituted alkyl, substituted or unsubstituted aryl, substituted or unsubstituted arylalkyl, substituted or unsubstituted heteroalkyl, substituted or unsubstituted heteroaryl, or Rₐ and R_{b} together with the atom to which they are attached form a heterocycloalkyl group; and
PG is a protecting group.

19. The process according to claim 1, wherein the catalyst is a metal complex comprising a platinum group metal, preferably the catalyst is a metal complex comprising palladium.

20. The process according to claim 1, wherein the catalyst is a complex of a platinum group metal, preferably palladium, and has one or more ligands coordinated to the platinum group metal, preferably said catalyst is [Pd(RuPhos)(crotyl)Cl] or [Pd(dippf)Cl₂].

21-24. (canceled)

25. The process according to claim 1, wherein the Lewis acid is lithium chloride, lithium bromide or lithium iodide.

26-28. (canceled)

29. The process according to claim 1, wherein the compound of Formula I is provided by reacting a compound of Formula IV, 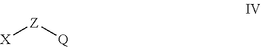
wherein X is a substituted or unsubstituted aromatic group;
Z is an organic group comprising an sp³-hybridised carbon atom C* and having formula —C*(H)(R³)— where R³ is H, OH, a substituted or unsubstituted alkyl group, a substituted or unsubstituted alkenyl group, or a substituted or unsubstituted aromatic group; and
Q is hydroxy, or —OM where M is a metal;
with a dialkylcarbonate of formula RO(CO)OR, wherein each R is a substituted or unsubstituted C₁₋₂₀ straight-chain or C₃₋₂₀ branched-chain alkyl, in the presence of a second base.

30. (canceled)

31. The process according to claim 1, wherein the compound of Formula I is provided in-situ in the presence of the compound of Formula II, the catalyst, the water, and the first base by reacting a compound of Formula IV 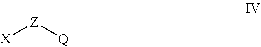
wherein X is a substituted or unsubstituted aromatic group;
Z is an organic group comprising an sp³-hybridised carbon atom C* and having formula —C*(H)(R³)— where R³ is H, OH, a substituted or unsubstituted alkyl group, a substituted or unsubstituted alkenyl group, or a substituted or unsubstituted aromatic group; and
Q is hydroxy, or —OM where M is a metal;
with a dialkylcarbonate of formula RO(CO)OR, wherein each R is a substituted or unsubstituted C₁₋₂₀ straight-chain or C₃₋₂₀ branched-chain alkyl group.

32-33. (canceled)

34. The process according to claim 1, wherein the Lewis acid is a metal halide or a lithium halide.

35. The process according to claim 1, wherein the Lewis acid is present in the range of from 1 to 4 molar equivalents relative to the compound of Formula I.
